(19) United States
(12) Patent Application Publication
(10) Pub. No.: US 2007/0249013 A1
(43) Pub. Date: Oct. 25, 2007

(54) Collections of Traceable Compounds and Uses Thereof

(76) Inventors: Marcel Francois Louis Hibert, Eschau (FR); Christel Anne Franchet, Hotzheim (FR); Galzi Jean-Luc, Weitbruch (FR); Franc Emile Jean Pattus, Strasbourg (FR); Fabrice Yves Guillier, Graffenstaden (FR)

Correspondence Address: YOUNG & THOMPSON, 745 SOUTH 23RD STREET, 2ND FLOOR, ARLINGTON, VA, 22202, US

(21) Appl. No.: 11/597,903

(22) PCT Filed: Jun. 15, 2005
(86) PCT No.: PCT/FR05/01502

§ 371 Date: Jun. 28, 2007

(30) Foreign Application Priority Data
Jun. 15, 2004 (FR) 0406462

(51) Int. Cl. G01N 33/58; C07K 2/00; C12Q 1/04; G01N 33/566

(52) U.S. Cl. 435/34; 436/501; 530/300

(57) ABSTRACT

The invention concerns the use of a collection of traceable cationic compounds for determining ligands of a receptor whose ligand is unknown or whose ligand useful for specific affinity binding studies is unknown, said traceable cationic compounds being characterized in that they comprise at least one basic amino acid residue providing the cationic type to said compound and a tracer group, in particular a fluorophor, a colouring agent or a quencher.

A subject of the present invention is collections of traceable compounds, as well as uses thereof, in particular within the framework of the determination of ligands of a receptor, no ligand of which is known or no ligand of which is known that can be used for specific affinity binding studies.

The gene coding for a fluorescent protein originating from the jelly fish Aequorea victoria, green fluorescent protein (or GFP) (Prasher et al. 1992, Gene 111 : 229-233), has recently been sequenced. GFP is a monomeric protein. It acquires its fluorescence properties by an autocatalytic fluorophore formation mechanism.

Numerous medicaments and natural substances perform their action by interacting with regulating proteins called receptors, involved in numerous physiological functions of organisms, and the alterations of their functions are the cause of numerous pathologies. The receptors' accessibility to natural or synthetic, endogenous or exogenous pharmacological agents from outside the cell leads to their being considered as targets of choice for research into biologically active molecules, in particular molecules having potential therapeutic powers.

The sequencing of the human genome gives access to the sequence of hundreds of novel proteins neither the endogenous ligand nor the biological function of which are known. These so-called “orphan” proteins, constitute potential sites for the action of medicaments.

The methods existing at present make it possible to detect only agonist molecules which activate a function associated with the protein. This type of functional test leads to numerous false positives which are costly in terms of time and money. Moreover, the antagonist molecules which would inhibit the function associated with the protein cannot be detected directly by these methods. Therefore, the antagonists have the best therapeutic potential most of the time.

The purpose of the present invention is to propose a method for the determination of any ligand, whether endogenous or exogenous, making it possible to dispense with a systematic search for the endogenous or natural ligand.

One of the aims of the invention is to provide a method for screening the first ligand of an orphan receptor without knowing its endogenous ligand.

The present invention relates to the use of a collection of traceable compounds of cationic type, for the determination of ligands of a receptor no ligand of which is known or no ligand of which is known that can be used for specific affinity binding studies, said traceable compounds of cationic type being characterized in that they include at least one amino acid residue which is basic in nature conferring the cationic type on said compound and a tracer group, in particular a fluorophore, a dye or a “quencher”.

The expression “traceable compounds of cationic type” designates molecules containing a group which is positively charged under physiological conditions.

The expression “receptor no ligand of which is known” designates any biological macromolecule (“receptor”) for which no endogenous or exogenous molecules binding to it in reversible manner are known.

The expression “specific affinity binding” designates the measurement of the interaction force between two molecules, namely a receptor and its ligand.

The expression “receptor no ligand of which is known that can be used for specific affinity binding studies” designates any biological macromolecule (receptor) for which no molecule yet exists which can bind to it (ligand) in a sufficiently specific and detectable manner.

The expression “amino acid residue which is basic in nature” designates an amino acid residue (alpha-, beta-, gamma-, delta-amino, or other) containing an entity which is protonable under physiological conditions.

The expression “tracer group” designates a fluorescent chemical entity or a chemical entity possessing properties of staining or absorption of certain wavelengths.

An advantageous use according to the invention is characterized in that the traceable compounds of cationic type comprise at least one “aa-cation-spacer-fluorophore” or “cation-aa-spacer-fluorophore” group, “aa” corresponding to an amino acid residue, “cation” corresponding to an amino acid residue which is basic in nature, “spacer” corresponding to a spacer group and “fluorophore” corresponding to a fluorophore group, said group being characterized in that the “aa” entity and the “cation” entity are linked to each other by a peptide or pseudo-peptide bond.

The expression “peptide bond” designates a —CO—NH— amide bond between two natural amino acids.

The expression “pseudo-peptide bond” designates a —CO—NH— bond between two non-natural amino acids.

The expression “spacer group” designates an entity separating the tracer group from the remainder of the molecule.

The present invention relates to the use of a collection of traceable compounds of cationic type corresponding to the following formula (I-1): 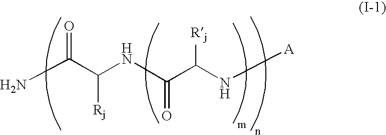
in which:
m is equal to 0 or 1,
n represents an integer varying from 1 to 10, and in particular from 1 to 6, and preferably being equal to 2 or 3,
i represents an integer varying from 1 to n,
Rⱼ and R′ⱼ represent an amino acid side chain, one at least of Rⱼ and R′ⱼ representing an amino acid side chain which is basic in nature, and, when m=1, at least two of the Rⱼ groups represent an amino acid side chain which is basic in nature and all the R′ⱼs are different from an amino acid side chain which is basic in nature,
A represents a tracer group, in particular a fluorophore, a dye or a “quencher”, or a group in the form D-G, D representing a spacer group and G representing a tracer group as defined previously,
   for the in vitro determination of ligands of a receptor, no ligand of which is known or no ligand of which is known that can be used for specific affinity binding studies.

An advantageous use according to the present invention is characterized in that the traceable compounds of cationic type correspond to the following formula (I): 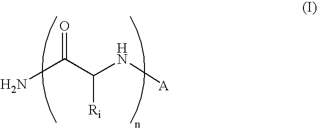
in which:
n represents an integer varying from 1 to 10, and in particular from 1 to 6, and preferably being equal to 2 or 3,
i represents an integer varying from 1 to n,
Rᵢ represents an amino acid side chain, at least one of the Rᵢs representing an amino acid side chain which is basic in nature, the Rᵢs being able to be all identical or different,
A represents a tracer group, in particular a fluorophore, a dye or a “quencher”, or a group in the form D-G, D representing a spacer group and G representing a tracer group as defined previously.

The present invention also relates to the salts of the traceable compounds of cationic type of the abovementioned formula (I).

The present invention also relates to the use as defined above, characterized in that the traceable compounds of cationic type correspond to the following formula: 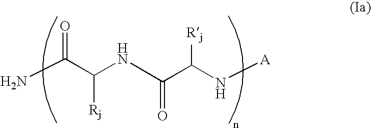
in which:
n represents an integer varying from 2 to 10, and in particular from 2 to 6, and preferably being equal to 2 or 3,
j corresponds to the definition given previously for i in the compounds of formula (I),
Rⱼ and R′ⱼ represent an amino acid side chain, characterized in that at least two of the Rⱼ groups represent an amino acid side chain which is basic in nature and in that all the R′ⱼs are different from an amino acid side chain which is basic in nature, the Rⱼs being able to be all identical or different and the R′ⱼs being able to be all identical or different, and
A is as defined above, and preferably represents a fluorophore.

The present invention also relates to a compound of the following formula (I-1): 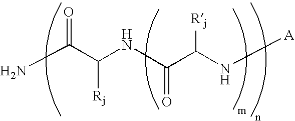
in which:
m is equal to 0 or 1,
n represents an integer varying from 1 to 10, and in particular from 1 to 6, and preferably being equal to 2 or 3,
i represents an integer varying from 1 to n,
Rⱼ and R′ⱼ represent an amino acid side chain, at least one of the Rⱼs and R′ⱼs representing an amino acid side chain which is basic in nature, and, when m=1, at least two of the Rⱼ groups represent an amino acid side chain which is basic in nature and all the R′ⱼs are different from an amino acid side chain which is basic in nature,
A represents a tracer group, in particular a fluorophore, a dye or a “quencher”, or a group in the form D-C, D representing a spacer group and G representing a tracer group as defined previously.

The present invention also relates to a compound of the following formula (I): 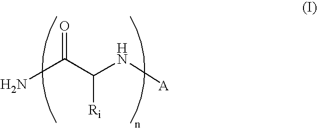
in which:
n represents an integer varying from 1 to 10, and in particular from 1 to 6, and preferably being equal to 2 or 3,
i represents an integer varying from 1 to n,
Rᵢ represents an amino acid side chain, at least one of the Rᵢs representing an amino acid side chain which is basic in nature, all the Rᵢs being able to be identical or different,
A represents a tracer group, in particular a fluorophore, a dye or a “quencher”, or a group in the form D-G, D representing a spacer group and G representing a tracer group as defined previously.

An advantageous compound according to the present invention is a compound of formula (I) characterized in that a single Rᵢ group represents an amino acid side chain which is basic in nature.

Such a compound corresponds to a traceable compound of monocationic type.

An advantageous compound according to the present invention is characterized in that it corresponds to the following formula: 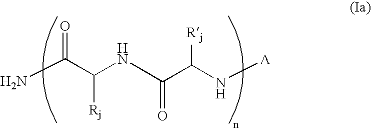
in which:
n represents an integer varying from 2 to 10, and in particular from 2 to 6, and preferably being equal to 2 or 3,
j corresponds to the definition given previously for i in claim 5,
Rⱼ and R′ⱼ represent an amino acid side chain, characterized in that at least two of the Rⱼ groups represent an amino acid side chain which is basic in nature and in that all the R′ⱼs are different from an amino acid side chain which is basic in nature, all the Rⱼs being able to be identical or different and all the R′ⱼs being able to be identical or different, and
A is as defined above.

A particularly advantageous compound according to the present invention is a compound of formula (Ia), characterized in that only two of the Rⱼ groups represent an amino acid side chain which is basic in nature.

Such compounds correspond to traceable compounds of biscationic type which have a strong probability of binding to certain classes of target receptors.

An advantageous compound according to the present invention is a compound of formula (Ia) as defined above, characterized in that:
Rⱼ represents an amino acid side chain which is basic in nature, and preferably the lysine, ornithine or arginine side chain,
R′ⱼ represents an amino acid side chain, said amino acid preferably being chosen from the group comprising: alanine, glycine, 6-aminocaproic acid, leucine, glutamine, glutamic acid, methionine, proline, isonipecotic acid, tetraisoquinoline carboxylic acid, 3-aminobenzoic acid, 4-aminomethylbenzoic acid, tryptophan, histidine, phenylalanine, tyrosine, 2-naphthylalanine, benzoyl phenylalanine.

According to a preferred embodiment, the present invention relates to a compound of formula (I), characterized in that n is equal to 2, and corresponding to the following formula (II): 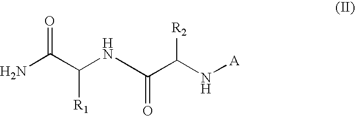
in which:
A is as defined above, and preferably represents a fluorophore,
R₁ represents an amino acid side chain which is basic in nature, and preferably the lysine, ornithine or arginine side chain, and
R₂ represents an amino acid side chain, said amino acid preferably being chosen from the group comprising: alanine, glycine, 6-aminocaproic acid, leucine, glutamine, glutamic acid, methionine, proline, isonipecotic acid, tetraisoquinoline carboxylic acid, 3-aminobenzoic acid, 4-aminomethylbenzoic acid, tryptophan, histidine, phenylalanine, tyrosine, 2-naphthylalanine, benzoyl phenylalanine.

The abovementioned compounds of formula (II) are monocationic compounds.

According to a preferred embodiment, the present invention relates to a compound of formula (I), characterized in that n is equal to 3, and corresponding to the following formula (III): 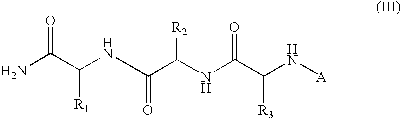
in which:
A is as defined above, and preferably represents a fluorophore,
R₁ and R₃ represent an amino acid side chain which is basic in nature, and preferably the lysine, ornithine or arginine side chain, and
R₂ represents an amino acid side chain, said amino acid preferably being chosen from the group comprising: alanine, glycine, 6-aminocaproic acid, leucine, glutamine, glutamic acid, methionine, proline, isonipecotic acid, tetraisoquinoline carboxylic acid, 3-aminobenzoic acid, 4-aminomethylbenzoic acid, tryptophan, histidine, phenylalanine, tyrosine, 2-naphthylalanine, benzoyl phenylalanine.

The abovementioned compounds of formula (III) are biscationic compounds.

An advantageous compound according to the present invention is a compound as defined above, characterized in that the spacer group D is chosen from the groups of the following formula:

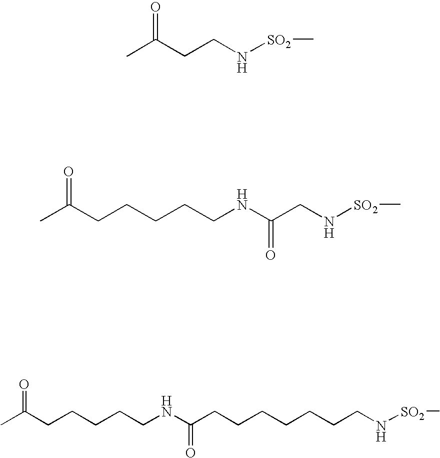

The present invention also relates to a compound as defined above, of formula (I), (Ia), (II) or (III), characterized in that A represents a fluorophore group the absorption and emission wavelengths of which are compatible with the fluorescence resonance energy transfer method with various green fluorescent protein mutants, said fluorophore being in particular chosen from the group comprising Bodipy and lissamine.

A preferred compound of the invention is a compound as defined above, of formula (I), (Ia), (II) or (III), characterized in that A represents one of the following groups:

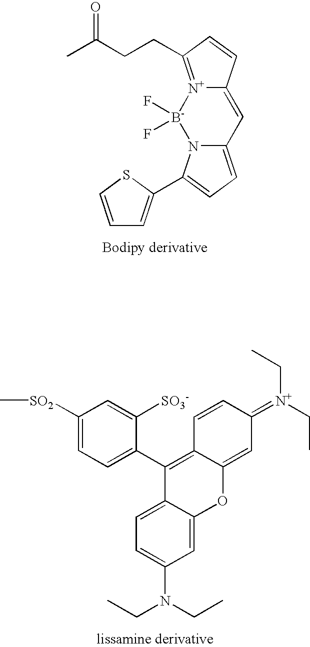

The present invention also relates to a collection comprising a plurality of compounds as defined above.

The expression “collection” designates a set of molecules prepared according to the same protocol.

The present invention also relates to the use of a collection as defined above, for the determination of ligands of a receptor no ligand of which is known or no ligand of which is known that can be used for specific affinity binding studies.

The present invention also relates to a method for screening ligands of receptors no ligand of which is known or no useable ligand of which is known, said method comprising the following stages:
bringing a collection of traceable compounds according to the invention together with cells transfected by a construction containing the fusion of the sequence coding for a fluorescent protein with the nucleotide sequence coding for a receptor no ligand of which is known or no useable ligand of which is known, and the mixture of said cells and of said collection,
detection of the fluorescence of said mixture, by excitation of said fluorescent protein and measurement of the emission fluorescence of said fluorescent protein, and determination of the fluorescence extinction percentage by comparing the emission fluorescence of said fluorescent protein in the mixture to the average fluorescence of said fluorescent protein in the absence of ligand,

the average fluorescence of said fluorescent protein in the absence of ligand being measured by control tests corresponding to the measurement of the fluorescence of the fluorescent protein in the absence of the collection of compounds, and
determination of the compounds which produce a fluorescence extinction percentage of the fluorescent protein of at least 5% and their identification as ligand.

The present invention relates more particularly to a method for screening ligands of G-protein-coupled receptors (GPCR) such as GPR50, GPR37, GPR19, GPR15, GPR31, GPR81, GPR3 and EDG7, the receptor APJ, FPRL1, the chemokine receptors CXCR1, CXCR2, CXCR3, CXCR4, the Glucagon-like peptide receptors (GLP-1R and GLP-2R), the CRF1 and CRF2 receptors, the metabotropic glutamate receptors (mGluR) and the GABA receptors.

The present invention also relates to a method for screening GPCR ligands no ligand of which is known, such as the orphan receptors GPR50, GPR37, GPR19, GPR31, GPR81, GPR3 and EDG7.

The present invention also relates to a method for screening GPCR ligands no ligand of which is known that can be used according to the method of the invention, such as the APJ receptor, the FPRL1 receptor, the chemokine receptors, CXCR1, CXCR2, CXCR3, CXCR4, the Glucagon-like peptide receptors (GLP-1R and GLP2-R), CRF1, CRF2, the metabotropic glutamate receptors (mGluR) and the GABA receptors.

The present invention relates to a preparation method on solid support of a compound as defined above, characterized in that it comprises the following stages:

a) a stage of coupling of the amine function of said solid support of the following formula: 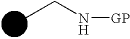
with a first amino acid with side chain R₁ the amine function of which is suitably protected by a protective group, in particular chosen from: Fmoc, Boc and Cbz, and preferably an Fmoc group, R₁ corresponding to the definition given previously for Rᵢ, in order to obtain a compound of the following formula:

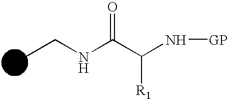

b) a stage of deprotection of the GP group under appropriate conditions, in order to obtain the compound of the following formula:

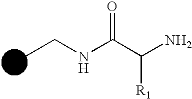

c) the sequential repetition of stages a) and b) until n amino acids have been grafted onto said solid support, which leads to the obtaining of the compound of the following formula:

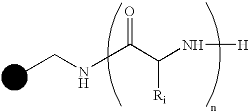

each sequence corresponding to
a stage of coupling a) of a compound of the following formula: 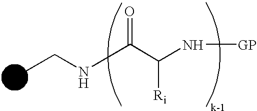
   k being an integer between 1 and n, with an amino acid with side chain Rₖ of formula 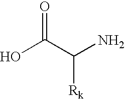
   the amine function of which is suitably protected, in order to obtain a compound of the following formula: 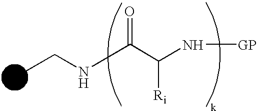
a stage of deprotection b) of the GP group under appropriate conditions, in order to obtain the compound of the following formula:

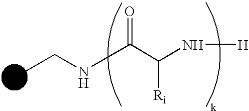

d) a stage of reaction of the compound obtained on completion of the abovementioned sequential repetition of formula 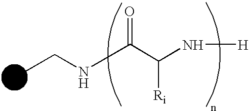
with a compound of formula A-W, A being as defined above and W representing a halogen atom or any nucleofugal group making it possible to activate an acid function and make it more reactive vis-à-vis the amines,

in order to obtain a compound of the following formula:

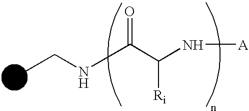

e) a stage of cleavage of the compound obtained in the preceding stage in order to obtain a compound of formula (I).

### DESCRIPTION OF THE FIGURES

FIG. 1 represents an emission spectrum of HEK 293 cells transfected by the construction pCEP4-SP-EGFP-GPR50. The y-axis corresponds to the wavelength (in nm) and the x-axis to the fluorescence intensity (in cps). The solid-line curve corresponds to the HEK cells expressing pCEP4-SP-EGFP-GPR50 and the dotted-line curve to the non-transfected HEK cells.

FIG. 2 represents the measurement in real time of the energy transfer between the fluorescent peptide ligands below (CP1 and CP2; 1 μM) and the GPR50-EGFP receptor. The time in seconds is shown along the x-axis and the fluorescence intensity at 510 nm (counts per second) along the y-axis, the excitation taking place at 470 nm (a low energy transfer of the order of 5% can be measured).

FIG. 3 represents an emission spectrum of HEK 293 cells transfected by the construction pcDNA6-SP-GFP-mGluR8. The y-axis corresponds to the wavelength (in nm) and the x-axis to the fluorescence intensity (in cps). The dotted-line curve corresponds to the HEK cells expressing pcDNA6-SP-GFP-mGluR8 and the solid-line curve to the non-transfected HEK cells.

### EXPERIMENTAL PART

Preparation of Fluorescent Tripeptides—General Diagram

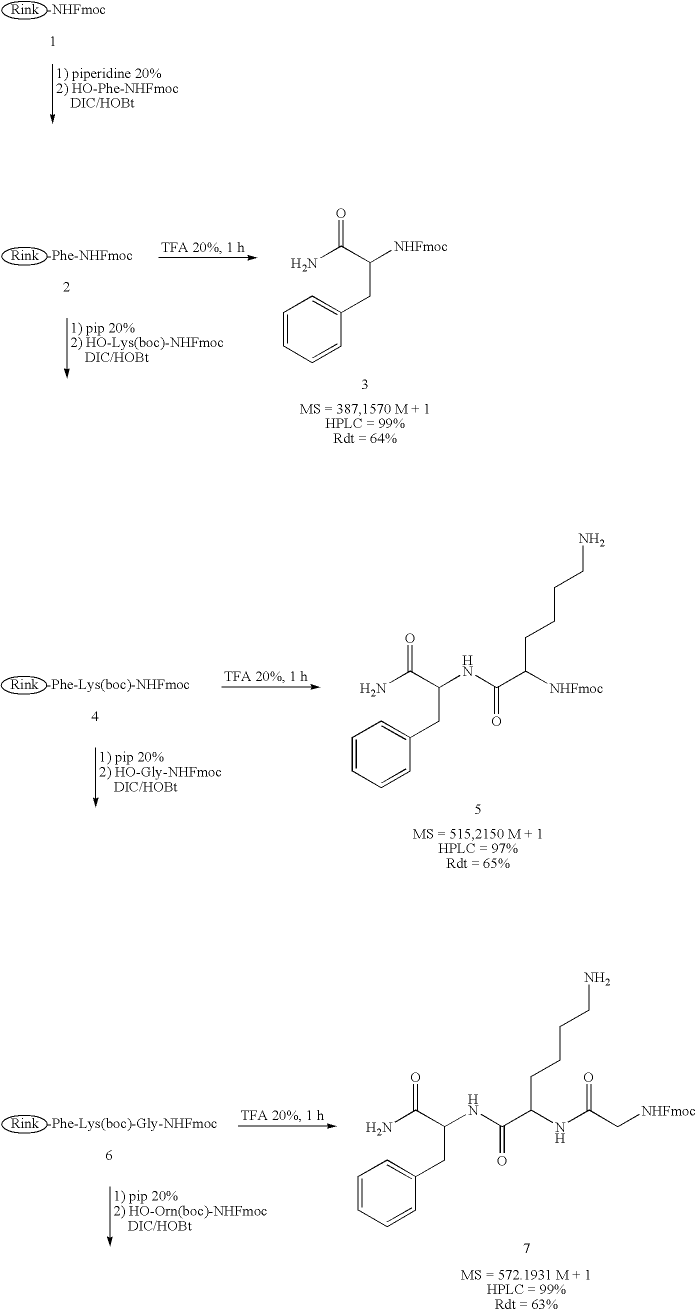

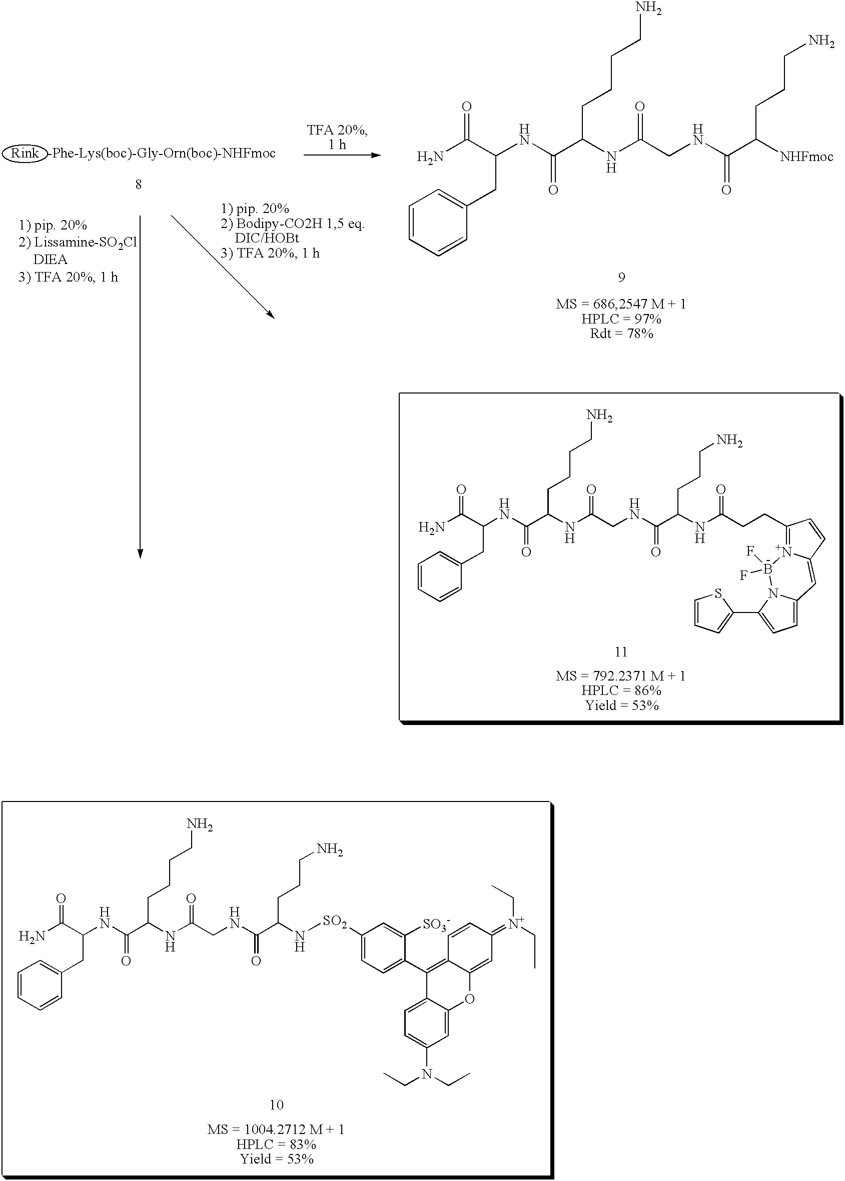

### EXPERIMENTAL PROTOCOL

I—General Procedures

1) General Procedure (A) for Grafting an Amino Acid onto a Rink Resin

Synthesis of the Rink-Phe-NHFmoc Resin (CHPO 72A or 116A; compound 1)

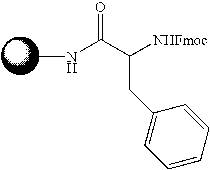

In a flask, the Rink-NHFmoc resin (5 g; 3 mmol; 0.6 mmol/g) is treated with 50 ml of a solution of 20% piperidine in DMF and stirred for 20 minutes. The mixture is filtered on frit and the resin is washed with two sequences of 50 ml of DMF, DCM, MeOH and one sequence of 50 ml of DMF, DCM. (CHPO 24A resin)

A solution of preactivated amino acid is prepared by mixing in the following order: N-α-Fmoc-L-phenylalanine (569 mg; 1.455 mmol) and HOBt (222 mg; 1.455 mmol) in 3 ml of DMF/DCM (1:1), for 15 minutes at ambient temperature. DIC (230 μl; 1.455 mmol) is added to the solution of carboxylic acid and HOBt and the mixture is stirred for 15 minutes at ambient temperature. During this time, the deprotected Rink-NH, resin CHPO 24A (700 mg; 0.485 mmol; 0.693 mmol/g) is expanded with 5 ml of DMF/DCM (1:4) in a flask, for 30 minutes. The preactivated carboxylic acid solution (1.455 mmol; 3 eq.) is added to the resin. The mixture is stirred for 14 hours at ambient temperature. The resin is filtered on frit, washed with two sequences of 10 ml of DMF, DCM, MeOH and one sequence of 10 ml of DMF, DCM, then dried under reduced pressure for 4 hours.

2) General Procedure (B) for Grafting Bodipy onto Rink Resin

Synthesis of the Rink-Phe-Bodipy Resin (CHPO 78A)

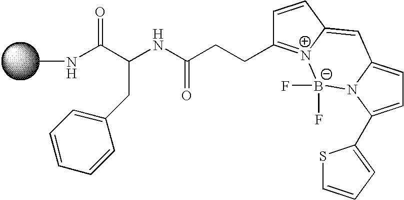

In a Supelco syringe equipped with a frit, the Rink-Phe-NHFmoc resin (677 mg; 0.374 mmol; 0.552 mmol/g) is treated with 7 ml of a solution of 20% piperidine in DMF and rotationally stirred for 20 minutes. The mixture is filtered and the resin is washed with two sequences of 7 ml of DMF, DCM, MeOH and one sequence of 7 ml of DMF, DCM (CHPO 77A resin).

A preactivated carboxylic acid solution is prepared by mixing in the following order: 4,4-difluoro-5-(2-thienyl)-4-bora-3a,4a-diaza-s-indacene-3-propionic acid CHPO65C (52 mg; 0.151 mmol) and HOBt (23 mg; 0.151 mmol) in 1 ml of DMF/DCM (1:1), for 15 minutes at ambient temperature. DIC (24 μl; 0.151 mmol) is added to the solution of carboxylic acid and HOBt and the mixture is stirred for 15 minutes at ambient temperature. During this time, the deprotected Rink-Phe-NH₂ resin CHPO 77A (200 mg; 0.126 mmol; 0.629 mmol/g) is expanded for 30 minutes with 2 ml of DMF/DCM (1:4) in a Supelco syringe equipped with a frit. The preactivated carboxylic acid solution (0.151 mmol; 1.2 eq.) is added to the resin. The mixture is rotationally stirred for 14 hours at ambient temperature. The resin is filtered through a frit, washed with two sequences of 3 ml of DMF, DCM, MeOH and one sequence of 3 ml of DMF, DCM, then dried under reduced pressure for 4 hours.

3) General Procedure (C) for the Cleavage of Fluorescent Molecules

Synthesis of H₂N—CO-Phe-Bodipy (CHPO 79A)

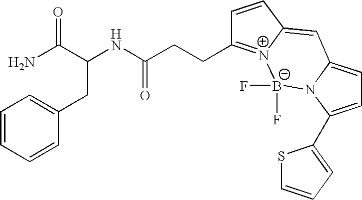

In a Supelco syringe, the Rink-Phe-Bodipy resin CHPO 78A (240 mg; 0.126 mmol; 0.521 mmol/g) is treated with 2 ml of TFA/DCM/H₂O (20:75:5) for 3 hours at ambient temperature. The resin is filtered, washed with DCM (3×3 ml). The filtrate is collected and taken to dryness under reduced pressure in order to produce 50 mg of intense violet solid.

Yield=80%, HPLC 81% (dionex, rt=29.59 min; λ=288 nm; gradient t=0 min: 2% of CH₃CN in H₂O (0.1% TFA) t=30 min: 100% CH₃CN t=40 min: 100%; 0.5 ml/min). MS (ESI-TOF) m/z (M+H) calculated for C₂₅H₂₃BF₂N₄O₂S+H, 493; found, 493. NMR ¹H (CDCl₃) δ 8.12-8.13 (m, 1H); 7.50-7.52 (m, 1H); 7.11-7.27 (m, 8H); 7.05-7.06 (m, 1H); 6.93-6.94 (m, 1H); 6.80-6.81 (m, 1H); 4.75-4.76 (m, 1H); 3.28-3.29 (m, 2H); 2.94-2.95 (m, 2H); 2.67-2.68 (m, 2H).

4) General Procedure (D) for Grafting Lissamine onto the Rink Resin

Synthesis of the Rink-Phe-Lissamine Resin (CHPO 112A)

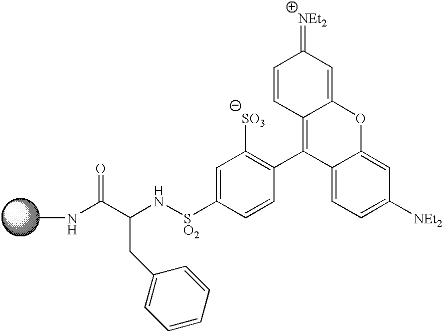

In a Supelco syringe equipped with a frit, the Rink-Phe-NHFmoc resin (630 mg; 0.350 mmol; 0.556 mmol/g) is treated with 7 ml of a solution of 20% piperidine in DMF and rotationally stirred for 20 minutes. The mixture is filtered and the resin is washed with two sequences of 7 ml of DMF, DCM, MeOH and one sequence of 7 ml of DMF, DCM. (CHPO 99B resin).

In a Supelco syringe equipped with a frit, the Rink-Phe-NH₂ resin (50 mg; 0.032 mmol; 0.635 mmol/g) is expanded with 1 ml of DCM. DIEA (11 μl; 0.064 mmol; 2 eq.) is added, then Lissamine rhodamine B sulphonyl chloride (37 mg; 0.064 mmol; 2 eq.) dissolved in 2 ml of DCM. The reaction mixture is stirred for 5 hours at ambient temperature. The resin is filtered, washed with two sequences of 3 ml of DMF, DCM, MeOH and one sequence of 3 ml of DMF, DCM, then dried under reduced pressure for 4 hours.

5) Synthesis of H₂N—CO-Phe-Lissamine (CHPO 112B)

(According to General Procedure (C))

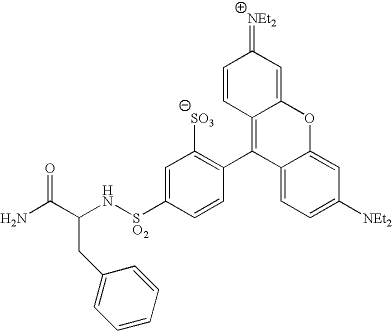

In a Supelco syringe, the Rink-Phe-lissamine resin CHPO 112A (20 mg; 0.042 mmol; 0.473 mmol/g) is treated with 2 ml of TFA/DCM/H₂O (20:75:5) for 2 hours 30 minutes at ambient temperature. The resin is filtered, washed with DCM (3×3 ml). The filtrate is collected and taken to dryness under reduced pressure in order to provide an intense violet solid.

HPLC 70% (dionex, rt=27.77 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₃₆H₄₀N₄O₇S₂+H, 705; found, 705.

6) Synthesis of the Rink-Arg(pmc)-NHFmoc resin (CHPO 74A)

(According to General Procedure (A))

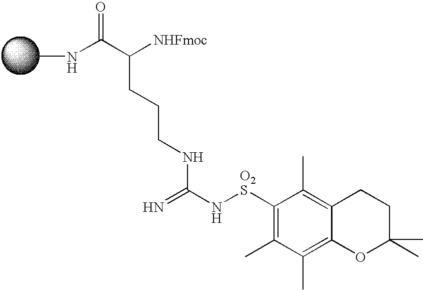

In a flask, the Rink-NHFmoc resin (5 g; 3 mmol; 0.6 mmol/g) is treated with 50 ml of a solution of 20% piperidine in DMF and stirred for 20 minutes. The mixture is filtered on frit and the resin is washed with two sequences of 50 ml of DMF, DCM, MeOH and one sequence of 50 ml of DMF, DCM (CHPO 24A resin).

A solution of preactivated amino acid is prepared by mixing in the following order: N-α-Fmoc-N-ω-(2,2,5,7,8-pentamethylchromane-6-sulphonyl)-L-Arginine (1.102 g; 1.663 mmol) and HOBt (254 mg; 1.663 mmol) in 10 ml of DMF/DCM (1:1), for 15 minutes at ambient temperature. DIC (262 μl; 1.663 mmol) is added to the solution of carboxylic acid and HOBt and the mixture is stirred for 15 minutes at ambient temperature. During this time, the deprotected Rink-NH₂ resin CHPO 24A (1.2 g; 0.832 mmol; 0.693 mmol/g) is expanded with 10 ml of DMF/DCM (1:4) in a flask, for 30 minutes. The preactivated carboxylic acid solution (1.455 mmol; 3 eq.) is added to the resin. The mixture is stirred for 14 hours at ambient temperature. The resin is filtered through a frit, washed with two sequences of 20 ml of DMF, DCM, MeOH and one sequence of 20 ml of DMF, DCM, then dried under reduced pressure for 4 hours.

7) Synthesis of the Rink-Arg(pmc)-Lissamine Resin (CHPO 113A)

(According to General Procedure (D))

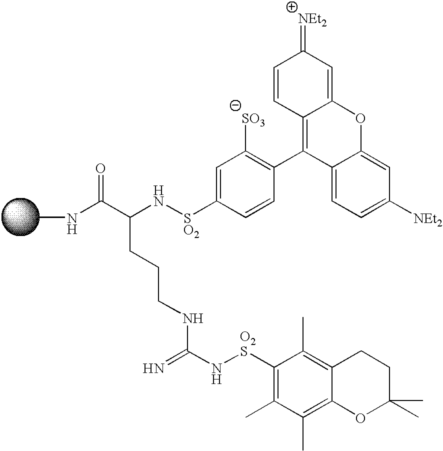

In a Supelco syringe equipped with a frit, the Rink-Arg(pmc)-NHFmoc resin CHPO 74A (852 mg; 0.408 mmol; 0.479 mmol/g) is treated with 7 ml of a solution of 20% piperidine in DMF and rotationally stirred for 20 minutes. The mixture is filtered and the resin is washed with two sequences of 7 ml of DMF, DCM, MeOH and one sequence of 7 ml of DMF, DCM (CHPO 80A resin).

In a Supelco syringe equipped with a frit, the Rink-Arg(pmc)-NH₂ resin CHPO80A (50 mg; 0.027 mmol; 0.536 mmol/g) is expanded with 1 ml of DCM. DIEA (9 μl; 0.054 mmol; 2 eq.) is added, then Lissamine rhodamine B sulphonyl chloride (31 mg; 0.054 mmol; 2 eq.) dissolved in 2 ml of DCM. The reaction mixture is stirred for 5 hours at ambient temperature. The resin is filtered, washed with two sequences of 3 ml of DMF, DCM, MeOH and one sequence of 3 ml of DMF, DCM, then dried wider reduced pressure for 4 hours.

8) Synthesis of H₂N—CO-Arg-Lissamine (CHPO 113B)

(According to General Procedure (C))

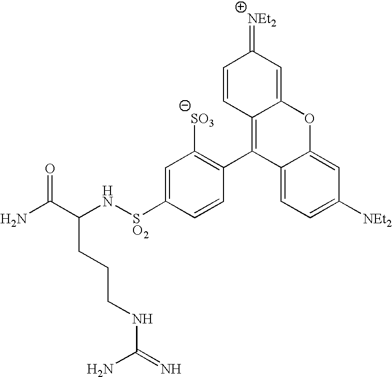

In a Supelco syringe, the Rink-Arg(pmc)-lissamine resin CHPO 113A (20 mg; 0.048 mmol; 0.416 mmol/g) is treated with 2 ml of TFA/DCM/H₂O (50:45:5) for 3 hours at ambient temperature. The resin is filtered, washed with DCM (3×3 ml). The filtrate is collected and taken to dryness under reduced pressure in order to provide an intense violet solid.

HPLC 80% (dionex, rt=24.47 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₃₃H₄₃N₇O₇S₂+H, 714; found, 714. NMR ¹H (CD₃OD) δ 8.64-8.66 (m, 1H); 8.14-8.17 (m, 1H); 7.52-7.56 (m, 1H); 6.97-7.56 (m, 6H); 3.93-3.99 (m, 1H); 3.76-3.79 (m, 8H); 3.23-3.26 (m, 2H); 1.65-1.80 (m, 4H); 2.02-2.11 (m, 12H).

II—Preparation of Fluorescent Cations

Synthesis of the Rink-Phe-NHFmoc Resin (CHPO 72A or 116A)

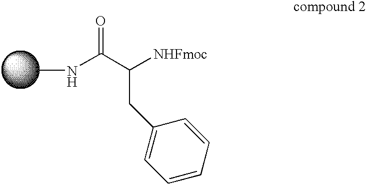

In a flask, the Rink-NHFmoc resin (5 g; 3 mmol; 0.6 mmol/g) (compound 1) is treated with 50 ml of a solution of 20% piperidine in DMF and stirred for 2 hours. The mixture is filtered on frit and the resin is washed with two sequences of 50 ml of DMF, DCM, MeOH and one sequence of 50 ml of DMF, DCM (CHPO 24A resin).

A solution of preactivated amino acid is prepared by mixing in the following order: N-α-Fmoc-L-phenylalanine (1.068 g; 2.76 mmol) and HOBt (422 mg; 2.76 mmol) in 20 ml of DMF/DCM (1:1), for 15 minutes at ambient temperature. DIC (435 μl; 2.76 mmol) is added to the solution of carboxylic acid and HOBt and the mixture is stirred for 15 minutes at ambient temperature. During this time, the deprotected Rink-NH₂ resin CHPO 24A (2.0 g; 1.38 mmol; 0.69 mmol/g) is expanded with 20 ml of DMF/DCM (1:4) in a flask, for 30 minutes. The preactivated carboxylic acid solution (2.76 mmol; 2 eq.) is added to the resin. The mixture is stirred for 14 hours at ambient temperature. The resin is filtered through a frit, washed with two sequences of 20 ml of DMF, DCM, MeOH and one sequence of 20 ml of DMF, DCM, then dried under reduced pressure for 4 hours.

Synthesis of H₂N—CO-Phe-NHFmoc (CHPO 116C)

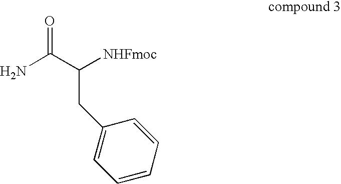

In a Supelco syringe, the Rink-Phe-NHFmoc resin CHPO 116A (compound 2) (80 mg; 0.145 mmol; 0.55 mmol/g) is treated with 1 ml of TFA/DCM/H₂O (20:75:5) for 2 hours at ambient temperature. The resin is filtered, washed with DCM (3×3 ml). The filtrate is collected and taken to dryness under reduced pressure in order to produce 11 mg of white solid.

Yield=64%. HPLC 99% (dionex, rt=30.11 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₂₄H₂₂N₂O₃+H, 387; found, 387. NMR ¹H (CDCl₃) δ 7.78 (d; J=7.5 Hz; 2H); 7.55 (dd; J=7.2 Hz; J=4.9 Hz; 2H); 7.42 (dd; J=7.5 Hz; J=7.1 Hz; 2H); 7.24-7.35 (m, 7H); 5.29-5.31 (m, 2H); 4.43-4.45 (m, 3H); 4.20 (t; J=6.4Hz; 1H); 3.04-3.12 (m, 2H).

Synthesis of the Rink-Phe-Lys(boc)-NHFmoc Resin (CHPO 120A)

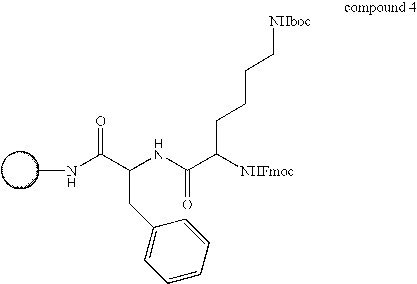

In a flask, the Rink-Phe-NHFmoc resin CHPO 116A (compound 2) (1.1 g; 0.605 mmol; 0.55 mmol/g) is treated with 10 ml of a solution of 20% piperidine in DMF and stirred for 2 hours. The mixture is filtered on frit and the resin is washed with two sequences of 10 ml of DMF, DCM, MeOH and one sequence of 10 ml of DMF, DCM (CHPO 118A resin).

A solution of preactivated amino acid is prepared by mixing in the following order: N-α-Fmoc-N-ω-terbutyloxycarbonyl-L-lysine (503 mg; 1.075 mmol) and HOBt (164 mg; 1.075 mmol) in 5 ml of DMF/DCM (1:1), for 15 minutes at ambient temperature. DIC (170 μl; 1.075 mmol) is added to the solution of carboxylic acid and HOBt and the mixture is stirred for 15 minutes at ambient temperature. During this time, the deprotected Rink-Phe-NH₂ resin CHPO 118A (857 mg; 0.537 mmol; 0.627 mmol/g) is expanded with 10 ml of DMF/DCM (1:4) in a flask, for 30 minutes. The preactivated carboxylic acid solution (1.075 mmol; 2 eq.) is added to the resin. The mixture is stirred for 14 hours at ambient temperature. The resin is filtered through a frit, washed with two sequences of 10 ml of DMF, DCM, MeOH and one sequence of 10 ml of DMF, DCM, then dried under reduced pressure for 4 hours.

Synthesis of H₂N—CO-Phe-Lys-NHFmoc (CHPO 120C)

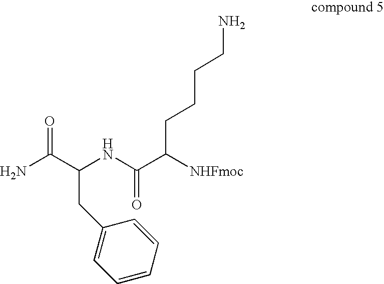

In a Supelco syringe, the Rink-Phe-Lys(boc)-NHFmoc resin CHPO 120A (compound 4) (80 mg; 0.163 mmol; 0.49 mmol/g) is treated with 1 ml of TFA/DCM/H₂O (20:75:5) for 1 hour at ambient temperature. The resin is filtered, washed with DCM (3×3 ml). The filtrate is collected and taken to dryness under reduced pressure in order to produce 13 mg of white solid.

Yield=65%. HPLC 97% (dionex, rt=25.93 min; λ=288 nm; gradient t: 0 min: 2% of CH₃CN in H₂O (0.1% TFA) t=30 min: 100% CH₃CN t=40 minutes: 100%; 0.5 ml/min). MS (ESI-TOF) m/z (M+H) calculated for C₃₀H₃₄N₄O₄+H, 515; found, 515. NMR ¹H (CD₃OD) δ 7.78 (d; J=7.5 Hz; 2H); 7.64-7.66 (m, 2H); 7.41 (dd; J=7.2 Hz; J=7.1 Hz; 2H); 7.32 (dd; J=7.2 Hz; J=6.8 Hz; 2H); 7.15-7.22 (m, 5H); 4.63-4.65 (m, 1H); 4.38-4.41 (m, 2H); 4.21-4.22 (m, 1H); 4.01-4.02 (m, 1H); 2.84-3.17 (m, 4H); 1.55-1.65 (m, 4H); 1.27-1.34 (m, 2H). NMR ¹³C (CD₃OD) δ 189.03; 157.63: (CO); 141.60; 137.18: C quat, arom; 131.40; 129.33; 128.41; 127.18; 126.74; 125.15; 119.96: CH arom, 64.35; 55.32; 54.43: CH, 66.98; 39.41; 37.71; 31.24; 26.99; 22.47: CH₂.

Synthesis of the Rink-Phe-Lys(boc)-Gly-NHFmoc Resin (CHPO 125A)

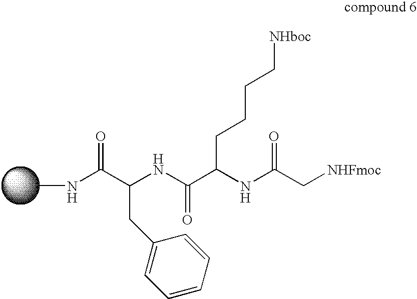

In a Supelco syringe, the Rink-Phe-Lys(boc)-NHFmoc resin CHPO 120A (compound 4) (970 mg; 0.475 mmol; 0.49 mmol/g) is treated with 10 ml of a solution of 20% piperidine in DMF and stirred for 2 hours. The mixture is filtered on frit and the resin is washed with two sequences of 10 ml of DMF, DCM, MeOH and one sequence of 10 ml of DMF, DCM (CHPO 122A resin).

A solution of preactivated amino acid is prepared by mixing in the following order: N-α-Fmoc-Glycine (261 mg; 0.88 mmol) and HOBt (134 mg; 0.88 mmol) in 5 ml of DMF/DCM (1:1), for 15 minutes at ambient temperature. DIC (138 μl; 0.88 mmol) is added to the solution of carboxylic acid and HOBt and the mixture is stirred for 15 minutes at ambient temperature. During this time, the deprotected Rink-Phe-Lys(boc)-NH₂ resin CHPO 122A (800 mg; 0.44 mmol; 0.55 mmol/g) is expanded with 5 ml of DMF/DCM (1:4) in a Supelco syringe, for 30 minutes. The preactivated carboxylic acid solution (0.88 mmol; 2 eq.) is added to the resin. The mixture is rotationally stirred for 14 hours at ambient temperature. The resin is filtered, washed with two sequences of 10 ml of DMF, DCM, MeOH and one sequence of 10 ml of DMF, DCM, then dried under reduced pressure for 4 hours.

Synthesis of H₂N—CO-Phe-Lys-Gly-NHFmoc (CHPO 125B)

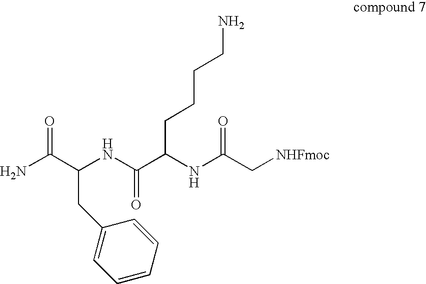

In a Supelco syringe, the Rink-Phe-Lys(boc)-Gly-NHFmoc resin CHPO 125A (compound 6) (100 mg; 0.210 mmol; 0.477 mmol/g) is treated with 1 ml of TFA/DCM/H₂O (20:75:5) for 1 hour at ambient temperature. The resin is filtered, washed with DCM (3×3 ml). The filtrate is collected and taken to dryness under reduced pressure in order to produce 17 mg of white solid.

Yield=63%. HPLC 99% (dionex, rt=25.54 min; λ=288 nm; gradient t: 0 min: 2% of CH₃CN in H₂O (0.1% TFA) t=30 minutes: 100% CH₃CN t=40 minutes: 100%; 0.5 ml/min). MS (ESI-TOF) m/z (M+H) calculated for C₃₂H₃₇N₅O₅+H, 572; found, 572. NMR ¹H (CD₃OD) δ 7.80 (d; J=7.2 Hz; 2H); 7.65 (d; J=7.2 Hz; 2H); 7.40 (dd; J=7.2 Hz; J=7.1 Hz; 2H); 7.15-7.34 (m, 7H); 4.59-4.62 (m, 1H); 4.37-4.39 (m, 2H); 4.24-4.25 (m, 2H); 3.74-3.78 (m, 2H); 3.22-3.30 (m, 1H); 2.95-3.00 (m, 1H); 2.80-2.83 (m, 2H); 1.50-1.60 (m, 4H); 1.27-1.31 (m, 2H). NMR ¹³C (CD₃OD) δ 172.61; 171.99: (CO); 150.14; 144.13; 137.66: C quat, arom, 129.26; 128.45; 127.83; 127.18; 126.74; 125.18; 119.96: CH arom, 54.77; 53.75; 47.30: CH; 67.34; 43.98; 39.40; 37.49; 34.94; 30.81; 26.89; 22.30: CH₂.

Synthesis of the Rink-Phe-Lys(boc)-Gly-Orn(boc)-NHFmoc Resin (CHPO 126A)

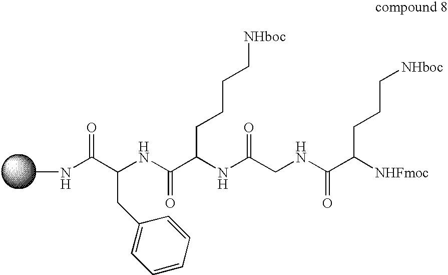

In a Supelco syringe, the Rink-Phe-Lys(boc)-Gly-NHFmoc resin CHPO 125A (compound 6) (740 mg; 0.353 mmol; 0.477 mmol/g) is treated with 5 ml of a solution of 20% piperidine in DMF and stirred for 2 hours. The mixture is filtered on frit and the resin is washed with two sequences of 5 ml of DMF, DCM, MeOH and one sequence of 5 ml of DMF, DCM (CHPO 125C resin).

A solution of preactivated amino acid is prepared by mixing in the following order: N-α-Fmoc-N-ω-terbutyloxycarbonyl-L-ornithine (281 mg; 0.619 mmol) and HOBt (83 mg; 0.619 mmol) in 3 ml of DMF/DCM (1:1), for 15 minutes at AT. DIC (98 μl; 0.619 mmol) is added to the solution of carboxylic acid and HOBt and the mixture is stirred for 15 minutes at ambient temperature. During this time, the deprotected Rink-Phe-Lys(boc)-Gly-NH₂ resin CHPO 125C (580 mg; 0.309 mmol; 0.534 mmol/g) is expanded with 2 ml of DMF/DCM (1:4) in a Supelco syringe, for 30 minutes. The preactivated carboxylic acid solution (0.88 mmol; 2 eq.) is added to the resin. The mixture is rotationally stirred for 14 hours at ambient temperature. The resin is filtered, washed with two sequences of 5 ml of DMF, DCM, MeOH and one sequence of 5 ml of DMF, DCM, then dried under reduced pressure for 4 hours.

Synthesis of H₂N—CO-Phe-Lys-Gly-Orn-NHFmoc (CHPO 126B)

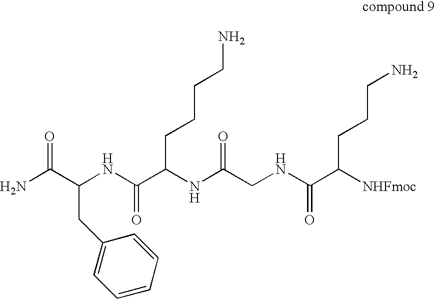

In a Supelco syringe, the Rink-Phe-Lys(boc)-Gly-Orn(boc)-NHFmoc resin CHPO 126A (compound 8) (80 mg; 0.184 mmol; 0.433 mmol/g) is treated with 1 ml of TFA/DCM/H₂O (20:75:5) for 1 hour at ambient temperature. The resin is filtered, washed with DCM (3×3 ml). The filtrate is collected and taken to dryness under reduced pressure in order to produce 18 mg of white solid.

Yield=78%. HPLC 97% (dionex, rt=23.37 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₃₇H₄₇N₇O₆+H, 686; found, 686. NMR ¹H (CD₃OD) δ 7.83 (d; J=7.2 Hz; 2H); 7.69 (d; J=7.2 Hz; 2H); 7.15-7.44 (m, 9H); 4.60-4.64 (m, 1H); 4.43-4.46 (m, 2H); 4.24-4.28 (m, 2H); 4.10-4.15 (m, 1H); 3.86-3.92 (m, 2H); 3.20-3.30 (m, 1H); 2.90-3.00 (m, 2H); 2.80-2.85 (m, 2H); 1.50-1.90 (m, 8H); 1.27-1.40 (m, 6H). NMR ¹³C (CD₃OD) δ 189.87; 175.06; 174.19; 172.72; 171.24; 157.71: (CO); 144.20; 141.63; 137.67: C quat, arom; 129.34; 128.43; 127.87; 127.21; 126.73; 125.17; 119.99: CH arom, 55.18; 54.78; 53.97; 48.86: CH; 67.16; 44.70; 39.37; 37.53; 30.7; 29.72; 28.95; 26.84; 24.02; 22.36: CH₂.

Synthesis of the Rink-Phe-Lys(boc)-Gly-Orn(boc)-Bodipy Resin (CHPO 129A)

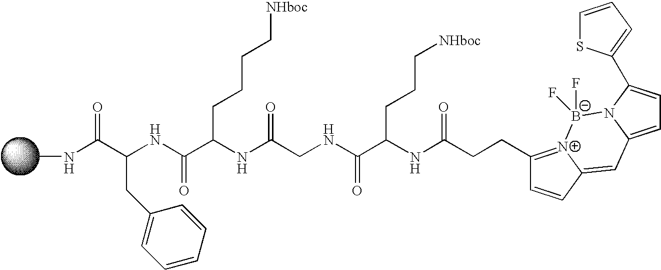

In a Supelco syringe, the Rink-Phe-Lys(boc)-Gly-Orn(boc)-NHFmoc resin CHPO 126A (compound 8) (625 mg; 0.27 mmol; 0.433 mmol/g) is treated with 5 ml of a solution of 20% piperidine in DMF and stirred for 2 hours. The mixture is filtered on frit and the resin is washed with two sequences of 5 ml of DMF, DCM, MeOH and one sequence of 5 ml of DMF, DCM (CHPO 127A resin).

A solution of preactivated carboxylic acid is prepared by mixing in the following order: 4,4-difluoro-5-(2-thienyl)-4-bora-3a,4a-diaza-s-indacene-3-propionic acid CHPO65C (20 mg; 0.0576 mmol) and HOBt (10 mg; 0.0768 mmol) in 3 ml of DMF/DCM (1:1), for 15 minutes at ambient temperature. DIC (12 μl; 0.0768 mmol) is added to the solution of carboxylic acid and HOBt and the mixture is stirred for 15 minutes at ambient temperature. During this time, the deprotected Rink-Phe-Lys(boc)-Gly-Orn(boc)-NH₂ resin CHPO 127A (80 mg; 0.0384 mmol; 0.48 mmol/g) is expanded with 2 ml of DMF/DCM (1:4) in a Supelco syringe, for 30 minutes. The preactivated carboxylic acid solution (0.0768 mmol; 2 eq.) is added to the resin. The mixture is rotationally stirred for 14 hours at ambient temperature. The resin is filtered, washed with two sequences of 5 ml of DMF, DCM, MeOH and one sequence of 5 ml of DMF, DCM, then dried under reduced pressure for 4 hours.

Synthesis of H₂N—CO-Phe-Lys-Gly-Orn-Bodipy (CHPO 129B)

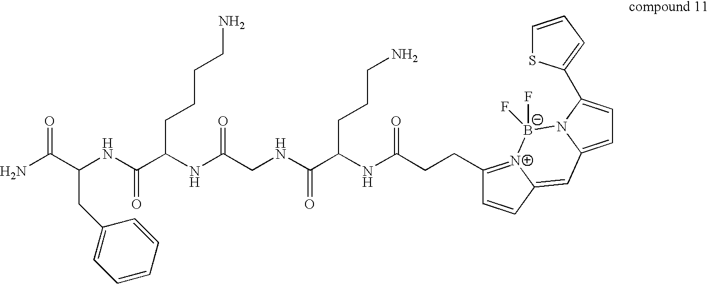

In a Supelco syringe, the Rink-Phe-Lys(boc)-Gly-Orn(boc)-Bodipy resin CHPO 129A (92 mg; 0.0384 mmol; 0.415 mmol/g) is treated with 1 ml of TFA/DCM/H₂O (20:75:5) for 1 hour at ambient temperature. The resin is filtered, washed with DCM (3×3 ml). The filtrate is collected and taken to dryness under reduced pressure in order to produce 14 mg of intense violet solid.

Yield=53%. HPLC 86% (dionex, rt=23.58 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₃₈H₄₈BF₂N₉O₅S+H, 792; found, 792. NMR ¹H (CD₃OD) δ 8.11 (d; J=3.8 Hz; 1H); 7.66 (d; J=4.9 Hz; 1H); 7.47 (s, 1H); 7.16-7.29 (m, 8H); 6.90 (d; J=4.5 Hz; 1H); 6.52 (d; J=3.8 Hz; 1H); 4.59-4.63 (m, 1H); 4.29-4.21 (m, 2H); 3.77-4.00 (m, 2H); 3.20-3.25 (m, 2H); 2.94-2.99 (m, 4H); 2.77-2.81 (m, 4H); 1.30-1.90 (m, 10H).

Compound 11 thus obtained is a compound of formula (Ia).

Synthesis of the Rink-Phe-Lys(boc)-Gly-Orn(boc)-Lissamine Resin (CHPO 130A)

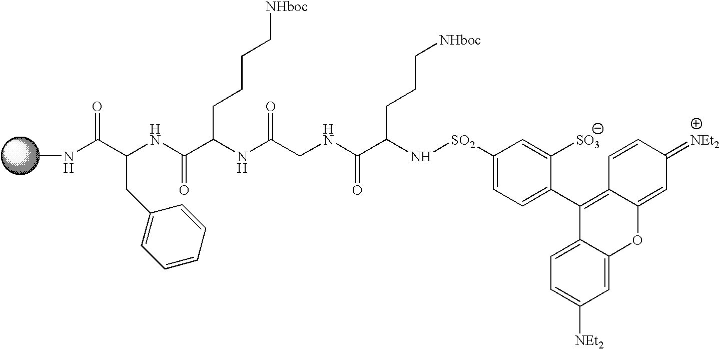

In a Supelco syringe, the Rink-Phe-Lys(boc)-Gly-Orn(boc)-NHFmoc resin CHPO 126A (compound 8) (625 mg; 0.27 mmol; 0.433 mmol/g) is treated with 5 ml of a solution of 20% piperidine in DMF and stirred for 2 hours. The mixture is filtered on frit and the resin is washed with two sequences of 5 ml of DMF, DCM, MeOH and one sequence of 5 ml of DMF, DCM (CHPO 127A resin).

In a Supelco syringe equipped with a frit, the Rink-Phe-Lys(boc)-Gly-Orn(boc)-NH₂ resin CHPO127A (80 mg; 0.0384 mmol; 0.48 mmol/g) is expanded with 1 ml of DCM/DMF (3:1). DIEA (13 μl; 0.0768 mmol; 2 eq.) is added, then Lissamine rhodamine B sulphonyl chloride (44 mg; 0.078 mmol; 2 eq.) dissolved in 3 ml of DCM/DMF (1:1). The reaction mixture is stirred for 5 hours at ambient temperature. The resin is filtered, washed with two sequences of 3 ml of DMF, DCM, MeOH and one sequence of 3 ml of DMF, DCM, then dried under reduced pressure for 4 hours.

Synthesis of H₂N—CO-Phe-Lys-Gly-Orn-Lissamine (CHPO 130B)

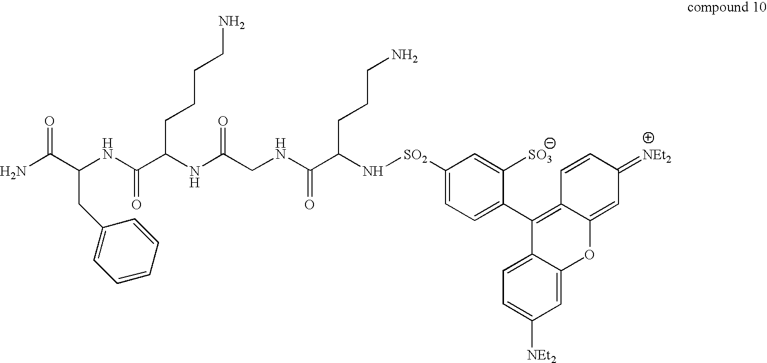

In a Supelco syringe, the Rink-Phe-Lys(boc)-Gly-Orn(boc)-lissamine resin CHPO 130A (100 mg; 0.0384 mmol; 0.38 mmol/g) is treated with 1 ml of TFA/DCM/H₂O (20:75:5) for 1 hour at ambient temperature. The resin is filtered, washed with DCM (3×3 ml). The filtrate is collected and taken to dryness under reduced pressure in order to produce 17 mg of intense violet solid.

Yield=53%. HPLC 83% (dionex, rt=23.76 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₄₉H₆₅N₉O₁₀S₂+H, 1004; found, 1004. NMR ¹H (CD₃OD) δ 8.64 (s, 1H); 8.18 (d; J=7.9 Hz; 1H); 7.55 (d; J=7.9 Hz; 1H); 6.97-7.28 (m, 11H); 4.61-4.65 (m, 1H); 4.12-4.14 (m, 1H); 3.96-4.00 (m, 1H); 3.67-3.70 (m, 8H); 3.20-3.30 (m, 1H); 2.95-3.05 (m, 4H); 2.82-2.90 (m, 4H); 1.21-1.86 (m, 22H). NMR ¹³C (CD₃OD) δ 174.89; 172.70; 170.40; 169.47: (CO); 161.48; 158.40; 156.37; 156.28; 156.13; 145.76; 142.54; 137.76; 137.46; 134.83; 114.27: C quat, arom,; 132.78; 131.83; 130.01; 129.47; 129.37; 128.72; 128.37; 126.78; 126.56; 96.04: CH arom, 56.69; 54.56; 52.80: CH; 45.80; 43.21; 39.40; 37.89; 31.15; 30.76; 29.74; 28.33; 26.98; 24.02; 22.91; 22.47: CH₂; 11.84: CH₃.

Compound 10 thus obtained is a compound of formula (Ia).

Other Example:

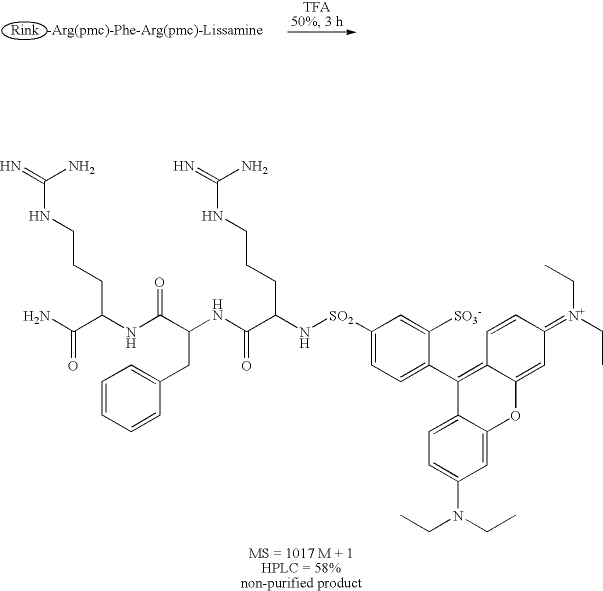

Synthesis of the Rink-Arg(pmc)-Phe-Arg(pmc)-Lissamine Resin (CHPO 133A)

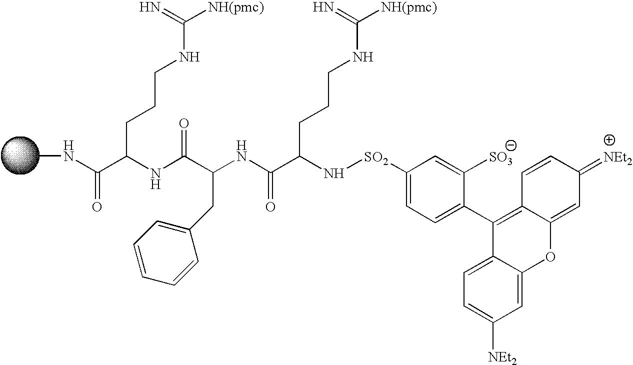

In a Supelco syringe, the Rink-Arg(pmc)-Phe-Arg(pmc)-NHFmoc resin CHPO 84A (803 mg; 0.317 mmol; 0.376 mmol/g) is treated with 5 ml of a solution of 20% piperidine in DMF and stirred for 2 hours. The mixture is filtered on frit and the resin is washed with two sequences of 5 ml of DMF, DCM, MeOH and one sequence of 5 ml of DMF, DCM (CHPO 84B resin).

In a Supelco syringe equipped with a frit, the Rink-Arg(pmc)-Phe-Arg(pmc)-NH₂ resin CHPO84B (100 mg; 0.041 mmol; 0.41 mmol/g) is expanded with 1 ml of DCM/DMF (3:1). DIEA (14 μl; 0.082 mmol; 2 eq.) is added, then Lissamine rhodamine B sulphonyl chloride (47 mg; 0.082 mmol; 2 eq.) dissolved in 3 ml of DCM/DMF (1:1). The reaction mixture is stirred for 5 hours at ambient temperature. The resin is filtered, washed with two sequences of 3 ml of DMF, DCM, MeOH and one sequence of 3 ml of DMF, DCM, then dried under reduced pressure for 4 hours.

Synthesis of H₂N—CO-Arg-Phe-Arg-Lissamine (CHPO 133B)

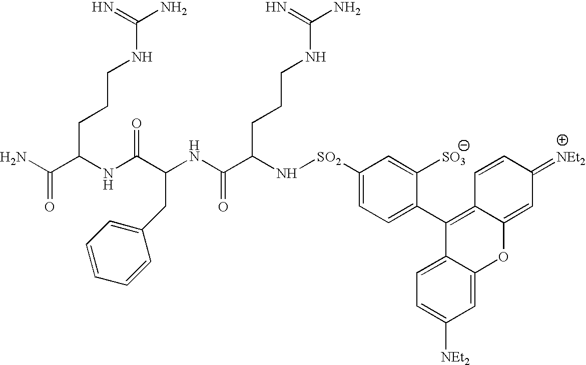

In a Supelco syringe, the Rink-Arg(pmc)-Phe-Arg(pmc)-lissamine resin CHPO 133A (122 mg; 0.041 mmol; 0.336 mmol/g) is treated with 1 ml of TFA/DCM/H₂O (50:45:5) for 3 hours at ambient temperature. The resin is filtered, washed with DCM (3×3 ml). The filtrate is collected and taken to dryness under reduced pressure in order to produce an intense violet solid.

HPLC 58% (dionex, rt=24.29 min; λ=288 nm; gradient t: 0 min: 2% of CH₃CN in H₂O (0.1% TFA) t=30 min: 100% CH₃CN t=40 min: 100%; 0.5 ml/min). MS (ESI-TOF) m/z (M+H) calculated for C₄₈H₆₄N₁₂O₉S₂+H, 1017; found, 1017.

The compound thus obtained corresponds to the general formula (III).

III—General Procedures for Plate Synthesis

General Procedure for Dispensing the Resin into a 96-Well Plate (CHPO 190)

0.007 mmol×96 wells×1.1 eq.=0.7392 mmol of Rink-NH, resin is placed in a beaker and expanded with 1 ml×96 wells×1.1 eq.=105.6 ml of DCM/DMF (1:1). The resin must be perfectly well distributed in the mixture of solvents. 1 ml of solution is distributed into each well, using a multichannel pipette.

General Procedure for Grafting an Amino Acid onto the Resin (CHPO 190)

A solution of preactivated amino acid is prepared in a pillbox by mixing in the following order: protected N-Fmoc amino acid (0.021 mmol) dissolved in 0.2 ml of DMF and HOBt (0.021 mmol) dissolved in 0.2 ml of DMF, for 15 minutes at ambient temperature. DIC (0.021 mmol) diluted in 0.3 ml of DMF is added to the solution of carboxylic acid and HOBt and the mixture is stirred for 15 minutes at ambient temperature. During this time, the deprotected NH₂ resin (0.007 mmol) is expanded in the wells with 0.2 ml of DMF, which is then filtered. The preactivated carboxylic acid solution (0.7 ml; 0.021 mmol; 3 eq.) is added to the resin, in each of the wells, using a multichannel pipette. 0.3 ml of DMF is added in order to complete the reaction. The plates are sealed by means of the Flexchem system and stirred for 14 hours at ambient temperature, on a Robbin device. On completion of the reaction, the plates are unsealed, the resin is filtered, washed with two sequences of 0.6 ml of DMF, DCM, MeOH and one sequence of 0.6 ml of DMF, DCM.

General Procedure for Grafting an Amino Acid onto the Resin (CHPO 191.)

The protected N-Fmoc resin (0.007 mmol) is expanded in the wells with 0.2 ml of DMF, which is then filtered. Then 1 ml of a solution of 20% piperidine in DMF is added to each well using a multichannel pipette. The plates are sealed by means of the Flexchem system and stirred for 3 hours at ambient temperature, on a Robbin device. On completion of the reaction, the plates are unsealed, the resin is filtered, washed with two sequences of 0.6 ml of DMF, DCM, MeOH and one sequence of 0.6 ml of DMF, DCM.

General Procedure for Grafting Lissamine (CHPO 198)

The resin (0.007 mmol) is expanded in the wells with 0.2 ml of DCM, which is then filtered. DIEA (0.014 mmol) diluted with 0.2 ml of DCM, then Lissamine-SO₂Cl (0.014 mmol) dissolved in 0.6 ml of DCM are added to each well, using a multichannel pipette. The wells are completed with 0.2 ml of DCM. The plates are sealed (ChemTuff joints, suitable for DCM) by means of the Flexchem system and stirred for 5 hours at ambient temperature, on a Robbin device. On completion of the reaction, the plates are unsealed, the resin is filtered, washed with DCM (2×0.6 ml), MeOH (1×0.6 ml) DMF (3×0.6 ml for 20 min), a sequence of 0.6 ml of DCM, MeOH, DMF, DCM, (DCM/DIEA 5%), DCM and finally DMF (5×0.6 ml).

General Procedure for Cleavage (CHPO 199)

The resin (0.007 mmol) is expanded in the wells with 0.2 ml of DCM, which is then filtered. 0.6 ml of a solution of TFA/DCM/H₂O (50:45:5) is added to each well, using a multichannel pipette. The plates are sealed by means of the Flexchem system and stirred for 3 hours at ambient temperature, on a Robbin device. On completion of the reaction, the plates are unsealed and placed on Deepwell plates in order to recover the filtrate during the filtration of the resin. The latter is filtered, washed with DCM (2×0.2 ml). The Deepwell plates are taken to dryness using a Genevac DD4. The resin is again washed with DMF (1×0.5 ml+2×0.25 ml), then the plates are taken to dryness using a Genevac.

### EXAMPLES OF SCREENING COLLECTIONS

### Example 1

### Expression of the Orphan Receptor GPR 50 Fused at its Amino-Terminal End to the EGFP Protein and Screening of Fluorescent Combinatorial Library

I) Fusion of the Coding Sequence of the Receptor GPR 50 to the EGFP

The coding sequence of the receptor GPR50 (Genbank accession No.: U 52219) is placed in the vector pCEP4-SP-EGFP. These 3′ and 5′ ends contain sequences complementary to the 5′ and 3′ ends of the vector pCEP4-SP-EGFP digested by Fse1. These complementary sequences were introduced into the primers used during the PCR (sequences underlined) in order to amplify the coding sequence of the receptor.

Sense primer: 5′ G GCC GGG GCC GGG ACC CCC TAT GGC TGT ATT GGC

Anti-sense primer: 5′ CTC GTT CTC GTT GGA TCA CAC AGC CAT TTC ATC AGG ATC

This method of directional insertion of PCR fragments into a vector, does not involve any restriction enzyme (Aslanidis C et al.; 1990, Nucleic Acids Res. 18, 6069-6074, Haun R. S. et al.; 1992, BioTechniques 13, 515-518).

In the construction pCEP4-SP-EGFP-GPR50, a spacer arm 6 amino acids in length separates the EGFP from the receptor. As for the receptor, it possesses a amino-terminal end 30 amino acids in length, which has been truncated by 9 amino acids relative to its wild-type sequence.

II) Expression of the Recombinant Proteins

HEK 293 cells are transfected by the calcium phosphate precipitation method (Chen & Okayama 1987, Mol. Cell. Biol. 7: 2745-2752) by the construction pCEP4-SP-EGFP-GPR50. Stable lines are established by selection of hygromycin-resistant transfected cells (500 μg/ml, Clontech). The cells are cultured in an MEM medium (Gibco) supplemented by 10% foetal calf serum (Sigma), penicillin (100 units/ml), streptomycin (100 μg/ml) and glutamine (4 mM).

III) Measurements of the Expression of the Receptor GPR50 Fused to EGFP, by Fluorimetry

The fluorescence experiments are carried out in a 1 ml cuvette provided with a magnetic stirring system and placed in a Fluorolog spectrofluorimeter (SPEX) equipped with an Xe 450 W lamp (Osram) and Spex 1680 0.22 m (excitation) and Spex 1681 0.22 m (emission) monochromators. The cells are suspended in a physiological buffer: Hepes: 0 mM, NaCl 137.5 mM, MgCl₂ 1.25 mM, CaCl₂ 1.25 mM, KCl 6 mM, glucose 5.6 mM, NaH₂PO₄ 0.4 mM, BSA 0.1% (W/v), pH 7.4.

The whole cells are harvested after treatment with trypsin-EDTA (1× Gibco) at ambient temperature. The cells are centrifuged for 5 minutes at 100 g and resuspended at a concentration of 1 to 2.106 cells/ml in the physiological buffer.

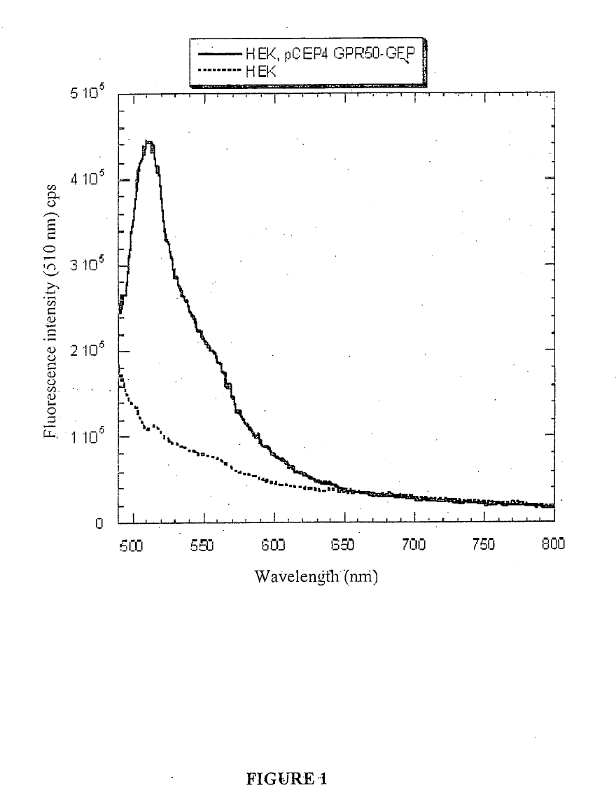

An emission spectrum of the cells transfected by pCEP4-SP-EGFP-GPR50 is shown in FIG. 1. A spectrum is produced before each screening. The spectra clearly show the EGFP signal, indicating that these cells express EGFP clearly in comparison with the non-transfected cells.

IV) Screening of Collections of Fluorescent Compounds on the HEK Line Expressing the Receptor GPR50 Fused to EGFP

IV.1. Screening Procedure

The distribution of ligands and cells is carried out using an automated pipetter-distributer (Biomek 2000) and the fluorescence is measured in a spectrofluorimeter (Victor II, Perkin Elmer).

98 μl of physiological buffer, 2 to 4 μl of fluorescent ligand and 100,000 cells (100 μl) of a cell suspension are distributed, chronologically, into a black 96-well plate, (Labsystem).

The fluorescent ligands are screened at a final concentration varying from 500 nM to 1 μM. These ligands are dissolved in DMSO, the final concentration of which in the test varies between 1 and 2%. The cells are distributed from a reservoir, in which the cells are kept in suspension by alternating aspiration and expulsion carried out by the automated device before each distribution. In order to mix all of the compounds, the robot aspirates and expels half of the wells once. The plate is then incubated for 10 minutes at ambient temperature, then centrifuged for 5 minutes at 100 g. It is then placed in the Victor fluorescence detector. The EGFP is excited at 465±7 nm and its emission is measured at 510±7 nm, for 2 seconds, 0.8 mm from the bottom of the plate.

In a 96-well plate, 16 wells serve as a control and 80 fluorescent ligands are tested, i.e. one per well. The emission of the EGFP (510 nm) from each of the wells treated is compared with the average fluorescence of the EGFP (510 nm) from the 16 control wells. A fluorescence extinction percentage is thus calculated and corresponds to the criterion of identification of the leads.

Three thousand fluorescent ligands were tested according to this protocol.

IV.2. Confirmation and Structure of the Potential Leads

The binding of certain molecules was verified with the spectrofluorimeter. The measurements are carried out in a 1 ml cuvette with a suspension of 1 to 2.106 cells/ml of physiological buffer.

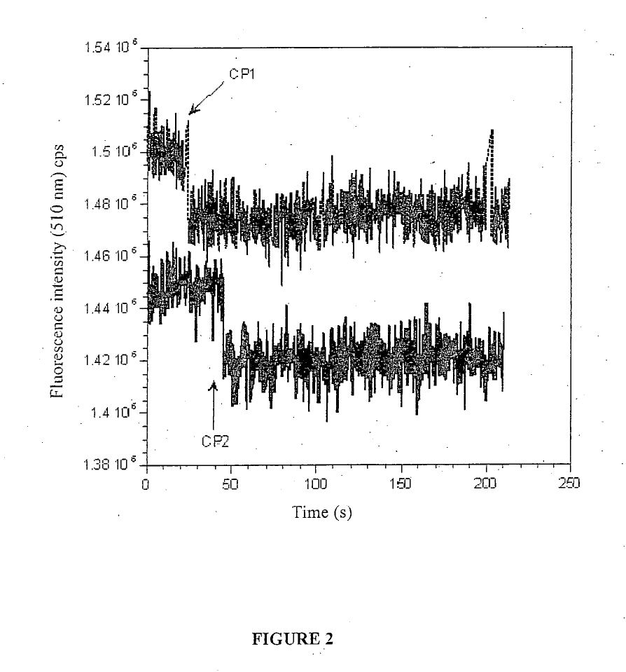

Two of the preferred ligands discovered by screening (see FIG. 2) possess the following structures:

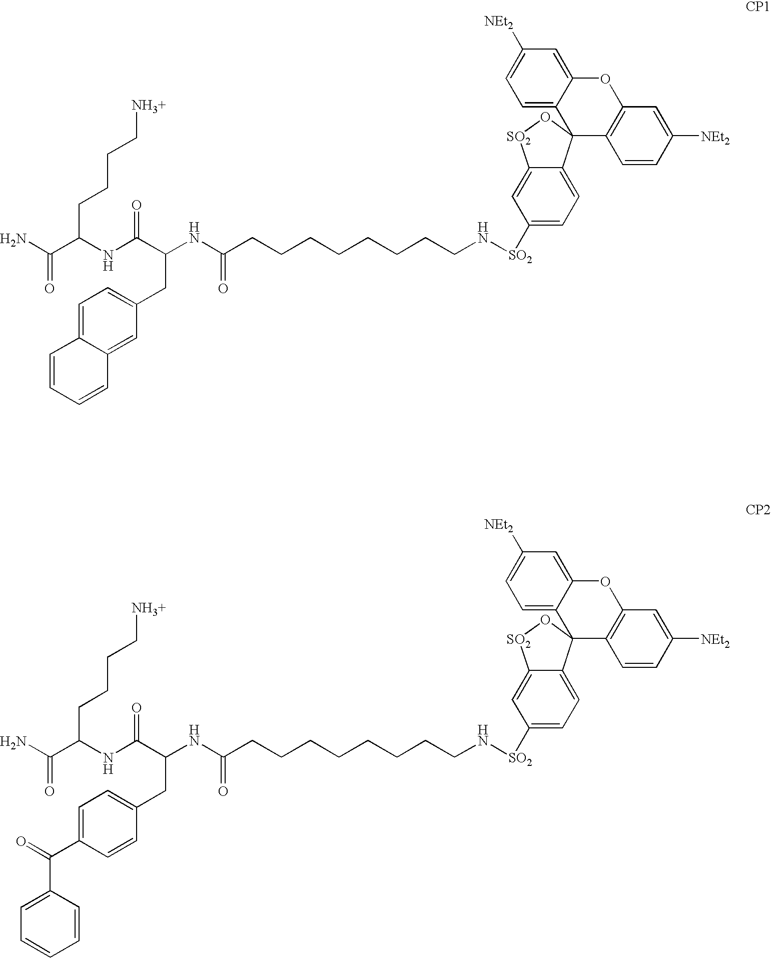

CP1 corresponds to a compound of the abovementioned formula (I) in which n=2, R₁ represents the lysine side chain, R₂ represents the 2-naphthylalanine side chain and A represents a group in the form D-G, D representing a spacer group of formula —CO—(CH₂)₈—NH—SO₂— and A representing a lissamine derivative.

CP2 corresponds to a compound of the abovementioned formula (I) in which n=2, R₁ represents the lysine side chain, R₂ represents the benzoyl phenylalanine side chain and A represents a group in the form D-G, D representing a spacer group of formula —CO—(CH₂)₈—NH—SO₂— and A representing a lissamine derivative.

### Example 2

### Expression of the mGluR8 Receptor Fused at its Amino-Terminal End to the GFP Protein and Screening of Fluorescent Combinatorial Libraries

I) Fusion of the Coding Sequence of the Receptor mGluR8 to GFP

The coding sequence of the subtype 8 metabotropic glutamate receptor (in GluR8) (Genbank accession No.: P70579) is placed in the vector pcDNA6. The 3′ and 5′ ends of the coding sequence contain sequences complementary to the 5′ and 3′ ends of the vector pcDNA6 digested by Fse1. These complementary sequences were introduced into the primers used during the PCR (underlined sequences) in order to amplify the coding sequence of the receptor.

The coding sequence of the receptor is amplified by PCR using the primers:

Sense primer:
5′ GGCCGCCCCCCGGGAGCCCCTGGGCTGTGGTACCT
Anti-sense primer:
5′ CTCGTTCTCGTTGGATTAGATCGAATGATTACTGTAGCTG

This sequence is placed downstream of the coding sequence of the GFP protein, which is itself placed downstream of the sequence coding for the peptide signal of the chicken alpha 7 subunit of the nicotinic acetylcholine receptor (corresponding to the amino acid sequence 1 to 25 of the protein sequence described in Genbank accession No.: X52295). The construction obtained is called pcDNA6-SP-GFP-mGluR8.

This method of directional insertion of PCR fragments into a vector does not involve any restriction enzyme (Aslanidis C et al.; 1990, Nucleic Acids Res. 18, 6069-6074, Haun R. S. et al.; 1992, BioTechniques 13, 515-518).

In the construction pcDNA6-SP-GFP-mGluR8, a spacer arm 6 amino acids in length separates GFP from the receptor. As for the receptor, its amino-terminal end composed of 550 amino acids has been truncated by 547 amino acids. In this construction, there are therefore 9 amino acids which separate GFP from the first transmembrane domain of the receptor.

II) Expression of Recombinant Proteins

HEK 293 cells are transfected by a lipofectamine 2000 transfection agent (Invitrogen) according to the supplier's instructions with the construction pcDNA6-SP-GFP-mGluR8. Stable lines are established by selection of the transfected blasticidin-resistant cells (5 μg/ml, Invivogen). The cells are cultured in an MEM medium (PAA) containing L-glutamine (2 mM) and supplemented with 10% foetal calf serum (PAA), penicillin (100 units/ml), streptomycin (100 μg/ml).

III) Measurements of the Expression of the mGluR8 Receptor Fused to GFP, by Fluorimetry

The fluorescence experiments are carried out in a 1 ml cuvette provided with a magnetic stirring system and placed in a Fluorolog-3 spectrofluorimeter (Jobin-Yvon Horiba). The cells are suspended in a physiological buffer: Hepes 10 mM; NaCl 137.5 mM; MgCl₂ 1.25 mM; CaCl₂ 1.25 mM; KCl 6 mM; glucose 5.6 mM; NaH₂PO₄ 0:4 mM; BSA 0.1% (W/v) pH 7.4.

The whole cells are harvested after treatment with PBS (PAA), EDTA 5 mM at ambient temperature. The cells are centrifuged for 5 minutes at 100 g and resuspended at a concentration of 1 to 2.10⁶ cells/ml in the physiological buffer.

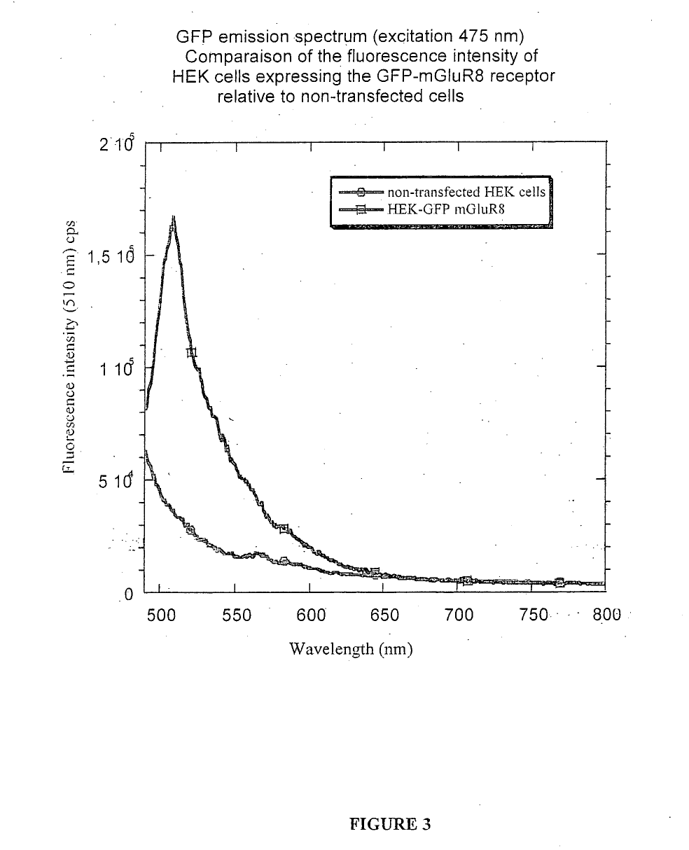

An emission spectrums of the cells transfected by the pcDNA-SP-GFP-mGluR8 is shown in FIG. 3. A spectrum is produced before each screening. The spectra clearly show the GFP signal, indicating that these cells express GFP clearly in comparison with the non transfected cells.

IV) Screening of Collections of Fluorescent Compounds on the HEK Line Expressing the Receptor mGluR8 Fused to GFP

IV.1. Screening Procedure

Twenty-four hours before the screening, the cells are seeded in 96-well plates. At the time of the experiment, the wells are rinsed out and the cells incubated in a physiological buffer the composition of which is described in paragraph III. The fluorescent ligands to be tested are distributed over the cells by the Flexstation (Molecular Devices) which follows the evolution of the fluorescence of GFP (excitation 475+/−8 nm, emission 510+/−8 nm) over time.

The fluorescent ligands are screened at a final concentration varying from 150 nM to 500 nM. These ligands are dissolved in DMSO, the final concentration of which in the test varies between 1 and 2%. A fluorescence extinction percentage of GFP is calculated for each well and corresponds to the criterion of identification of the leads.

Approximately 630 fluorescent ligands originating from the combinatorial library described in the present invention were tested according to this protocol.

### Example 3

### Expression of the Glucagon-Like Peptide-1 (GLP-1) Receptor Fused at its Amino-Terminal End to GFP Protein and Screening of Fluorescent Combinatorial Library

I) Fusion of the Coding Sequence of the GLP-1 Receptor to GFP

A fluorescent GLP-1R receptor is obtained by fusion of part of its coding sequence to that of GFP. The coding sequence of the GLP-1 receptor (Genbank accession No.: NM_{—}002062) is placed in a vector pcDNA6 downstream of the coding sequence of the autofluorescent GFP protein, which is itself placed downstream of the coding sequence for the peptide signal of the chicken alpha 7 subunit of the nicotinic acetylcholine receptor (corresponding to the amino acid sequence 1 to 25 of the sequence of the protein described in the Genbank accession No.: NP_{—}989512). In the construction thus obtained, called pCDNA6-SP-GFP-GLP1R, a spacer arm corresponding to the sequence coding for two amino acids separates GFP from the receptor. As for the receptor, a version truncated by 134 amino acids in its amino-terminal part was used for this study. This variant of the receptor possesses the characteristic of no longer allowing the binding of its endogenous ligand, the peptide related to Glucagon, GLP-1.

II) Expression of the Recombinant Proteins

HEK 293 cells are transfected by a lipofectamine 2000 transfection agent (Invitrogen) according to the supplier's instructions with the construction pcDNA6-SP-GFP-GLP-1R. Stable lines are established by selection of the blasticidin-resistant transfected cells (5 μg/ml, Invivogen). The cells are cultured in an MEM medium (PAA) containing L-glutamine (2 mM) and supplemented with 10% foetal calf serum (PAA), penicillin (100 units/ml) and streptomycin (100 μg/ml).

III) Measurements of the Expression of the GLP-1 Receptor Fused to GFP, by Fluorimetry

Measurements of the expression of the GLP-1 receptor fused to GFP by fluorimetry are carried out as described previously in Example 2.

IV) Screening of Collections of Fluorescent Compounds on the HEK Line Expressing the Receptor GLP1R Fused to GFP

The procedures for screening and confirmation of the leads were carried out as previously described in Example 2.

Approximately 630 fluorescent ligands obtained from the combinatorial library described in the present invention were tested according to this protocol.

1-18. (canceled)

19. A compound of the following formula (I-1): 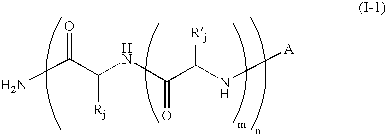
in which:
m is equal to 0 or 1,
n represents an integer varying from 1 to 10,
i represents an integer varying from 1 to n,
Rⱼ and R′ⱼ represent an amino acid side chain, at least one of the Rⱼs and R′ⱼs representing an amino acid side chain which is basic in nature, and, when m=1, at least two of the Rⱼ groups represent an amino acid side chain which is basic in nature and all the R′ⱼs are different from an amino acid side chain which is basic in nature,
A represents a tracer group, chosen from the group consisting of a fluorophore, a dye, and a “quencher”, or a group in the form D-G, D representing a spacer group and G representing a tracer group as defined previously.

20. A compound having the following formula (I): 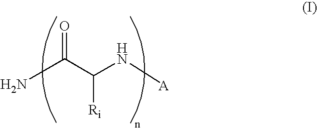
in which:
n represents an integer varying from 1 to 10,
i represents an integer varying from 1 to n,
Rᵢ represents an amino acid side chain, at least one of the Rᵢs representing an amino acid side chain which is basic in nature,
A represents a tracer group, chosen from the group consisting of a fluorophore, a dye, and a “quencher”, or a group in the form D-G, D representing a spacer group and G representing a tracer group as defined previously.

21. The compound of claim 20, characterized in that a single Rᵢ group represents an amino acid side chain which is basic in nature.

22. The compound of claim 19, characterized in that it corresponds to the following formula: 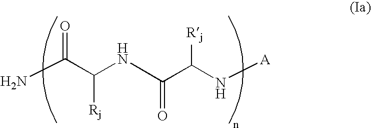
in which:
n represents an integer varying from 2 to 10,
j corresponds to the definition given previously for i,
Rⱼ and R′ⱼ represent an amino acid side chain, characterized in that at least two of the Rⱼ groups represent an amino acid side chain which is basic in nature and in that all the R′ⱼs are different from an amino acid side chain which is basic in nature, and
A is as previously defined.

23. The compound of claim 22, characterized in that only two of the Rⱼ groups represent an amino acid side chain which is basic in nature.

24. The compound of claim 22, characterized in that:
Rⱼ represents an amino acid side chain which is basic in nature, and is chosen from the group consisting of the lysine, the ornithine, and the arginine side chain,
R′ⱼ represents an amino acid side chain, said amino acid being chosen from the group consisting of: alanine, glycine, 6-aminocaproic acid, leucine, glutamine, glutamic acid, methionine, proline, isonipecotic acid, tetraisoquinoline carboxylic acid, 3-aminobenzoic acid, 4-aminomethylbenzoic acid, tryptophan, histidine, phenylalanine, tyrosine, 2-naphthylalanine, and benzoyl phenylalanine.

25. The compound according to claim 20, characterized in that n is equal to 2, and corresponding to the following formula (II): 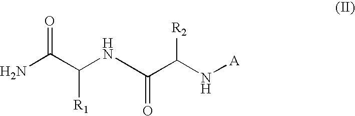
in which:
A is as previously defined,
R₁ represents an amino acid side chain which is basic in nature and is chosen from the group consisting of the lysine, the ornithine, and the arginine side chain, and
R₂ represents an amino acid side chain, said amino acid being chosen from the group consisting of: alanine, glycine, 6-aminocaproic acid, leucine, glutamine, glutamic acid, methionine, proline, isonipecotic acid, tetraisoquinoline carboxylic acid, 3-aminobenzoic acid, 4-aminomethylbenzoic acid, tryptophan, histidine, phenylalanine, tyrosine, 2-naphthylalanine, and benzoyl phenylalanine.

26. The compound according to claim 20, characterized in that n is equal to 3, and corresponding to the following formula (III): 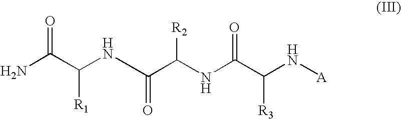
in which:
A is as previously defined,
R₁ and R₃ represent an amino acid side chain which is basic in nature, and is chosen from the group consisting of the lysine, the ornithine, and the arginine side chain, and
R₂ represents an amino acid side chain, said amino acid being chosen from the group consisting of: alanine, glycine, 6-aminocaproic acid, leucine, glutamine, glutamic acid, methionine, proline, isonipecotic acid, tetraisoquinoline carboxylic acid, 3-aminobenzoic acid, 4-aminomethylbenzoic acid, tryptophan, histidine, phenylalanine, tyrosine, 2-naphthylalanine, and benzoyl phenylalanine.

27. The compound according to claim 20, characterized in that the spacer group D is chosen from the groups of the following formula:

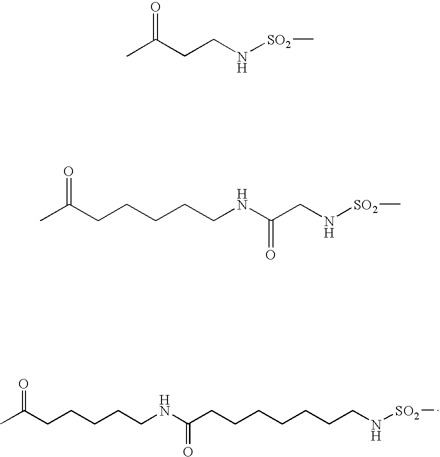

28. The compound according to claim 20, characterized in that A represents a fluorophore group the absorption and emission wavelengths of which are compatible with the fluorescence resonance energy transfer method with various green fluorescent protein mutants.

29. The compound according to claim 20, characterized in that A represents one of the following groups: 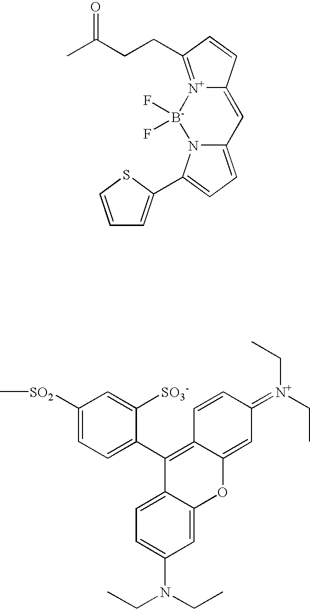
Bodipy derivative lissamine derivative

30. A collection comprising a plurality of compounds of formula (I) as defined in claim 20.

31. A method for screening ligands of receptors no ligand of which is known or no useable ligand of which is known, said method comprising the following stages:
bringing a collection of traceable compounds according to claim 30 together with cells transfected by a construction containing the fusion of the sequence coding for a fluorescent protein with the nucleotide sequence coding for a receptor no ligand of which is known or no useable ligand of which is known, and the mixture of said cells and of said collection,
detection of the fluorescence of said mixture, by excitation of said fluorescent protein and measurement of the emission fluorescence of said fluorescent protein, and determination of the fluorescence extinction percentage by comparing the emission fluorescence of said fluorescent protein in the mixture to the average fluorescence of said fluorescent protein in the absence of ligand,
the average fluorescence of said fluorescent protein in the absence of ligand being measured by control tests corresponding to the measurement of the fluorescence of the fluorescent protein in the absence of the collection of compounds, and
determination of the compounds which produce a fluorescence extinction percentage of the fluorescent protein of at least 5% and their identification as ligand.

32. A method for the preparation on solid support of a compound according to claim 20, characterized in that it comprises the following stages:
a) a stage of coupling of the amine function of said solid support of the following formula: 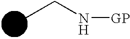
with a first amino acid with side chain R₁ the amine function of which is suitably protected by a protective group, R₁ corresponding to the previous definition for Rᵢ, in order to obtain a compound of the following formula: 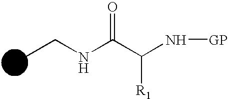
b) a stage of deprotection of the GP group under appropriate conditions, in order to obtain the compound of the following formula: 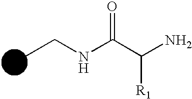
c) the sequential repetition of stages a) and b) until n amino acids have been grafted onto said solid support, which leads to the obtaining of the compound of the following formula: 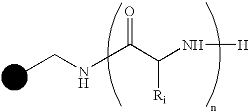
each sequence corresponding to
a stage of coupling a) of a compound of the following formula: 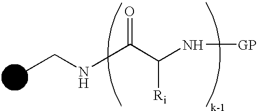
k being an integer between 1 and n,
with an amino acid with side chain Rₖ of formula 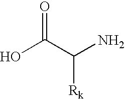
the amine function of which is suitably protected,
in order to obtain a compound of the following formula: 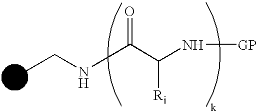
a stage of deprotection b) of the GP group under appropriate conditions, in order to obtain the compound of the following formula: 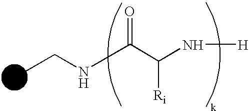
d) a stage of reaction of the compound obtained on completion of the abovementioned sequential repetition of formula 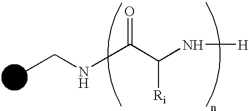
with a compound of formula A-W, A being as previously defined and W representing a halogen atom or any nucleofugal group making it possible to activate an acid function and make it more reactive vis-à-vis amines,
in order to obtain a compound of the following formula: 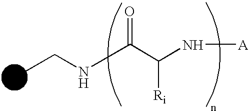
e) a stage of cleavage of compound obtained in the preceding stage in order to obtain a compound of formula (I).

33. The compound according to claim 22, characterized in that the spacer group D is chosen from the groups of the following formula:

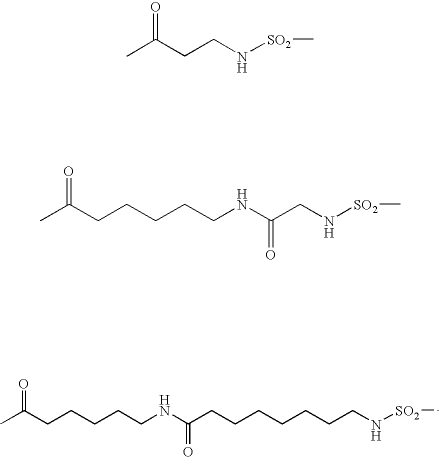

34. The compound according to claim 22, characterized in that A represents a fluorophore group the absorption and emission wavelengths of which are compatible with the fluorescence resonance energy transfer method with various green fluorescent protein mutants.

35. The compound according to claim 22, characterized in that A represents one of the following groups:

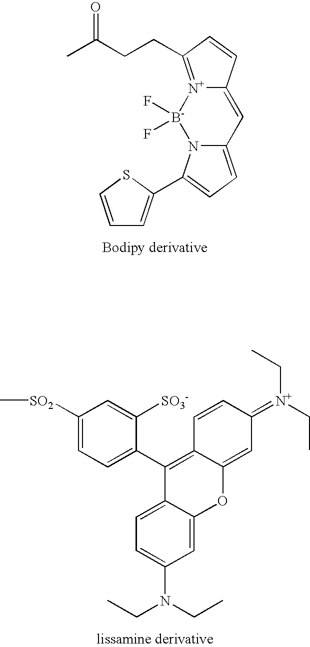

36. A collection comprising a plurality of compounds of formula (Ia) as defined in claim 22.

37. A method for screening ligands of receptors no ligand of which is known or no useable ligand of which is known, said method comprising the following stages:
bringing a collection of traceable compounds according to claim 36 together with cells transfected by a construction containing the fusion of the sequence coding for a fluorescent protein with the nucleotide sequence coding for a receptor no ligand of which is known or no useable ligand of which is known, and the mixture of said cells and of said collection,
detection of the fluorescence of said mixture, by excitation of said fluorescent protein and measurement of the emission fluorescence of said fluorescent protein, and determination of the fluorescence extinction percentage by comparing the emission fluorescence of said fluorescent protein in the mixture to the average fluorescence of said fluorescent protein in the absence of ligand,
the average fluorescence of said fluorescent protein in the absence of ligand being measured by control tests corresponding to the measurement of the fluorescence of the fluorescent protein in the absence of the collection of compounds, and
determination of the compounds which produce a fluorescence extinction percentage of the fluorescent protein of at least 5% and their identification as ligand.

38. A method for the in vitro determination of ligands of a receptor no ligand of which is known or no ligand of which is known that can be used for specific affinity binding studies, comprising the use of a collection of traceable compounds of cationic type corresponding to the following formula (I-1): 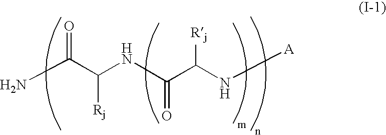
in which:
m is equal to 0 or 1,
n represents an integer varying from 1 to 10,
i represents an integer varying from 1 to n,
Rⱼ and R′ⱼ represent an amino acid side chain, one at least of Rⱼ and R′ⱼ representing an amino acid side chain which is basic in nature, and, when m=1, at least two of the Rⱼ groups represent an amino acid side chain which is basic in nature and all the R′ⱼs are different from an amino acid side chain which is basic in nature,
A represents a tracer group, chosen from the group consisting of: a fluorophore, a dye, and a “quencher”, or a group in the form D-G, D representing a spacer group and G representing a tracer group as defined previously.

39. A method for the in vitro determination of ligands of a receptor no ligand of which is known or no ligand of which is known that can be used for specific affinity binding studies, comprising the use of a collection of traceable compounds of cationic type having the following formula: 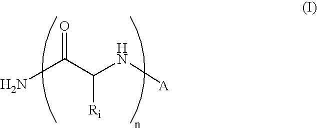
in which:
n represents an integer varying from 1 to 10,
i represents an integer varying from 1 to n,
Rᵢ represents an amino acid side chain, at least one of the Rᵢs representing an amino acid side chain which is basic in nature,
A represents a tracer group, chosen from the group consisting of: a fluorophore, a dye, and a “quencher”, or a group in the form D-G, D representing a spacer group and G representing a tracer group as defined previously.

40. A method for the in vitro determination of ligands of a receptor no ligand of which is known or no ligand of which is known that can be used for specific affinity binding studies, comprising the use of a collection of traceable compounds of cationic type having the following formula: 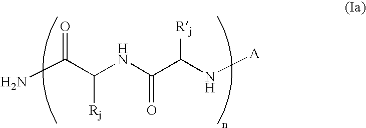
in which:
n represents an integer varying from 2 to 10,
j represents an integer varying from 1 to n,
Rⱼ and R′ⱼ represent an amino acid side chain, characterized in that at least two of the Rⱼ groups represent an amino acid side chain which is basic in nature and in that all the R′ⱼs are different from an amino acid side chain which is basic in nature, and
A represents a tracer group, chosen from the group consisting of: a fluorophore, a dye, and a “quencher”, or a group in the form D-G, D representing a spacer group and G representing a tracer group as defined previously.

41. A method for the in vitro determination of ligands of a receptor no ligand of which is known or no ligand of which is known that can be used for specific affinity binding studies, comprising the use of a collection of claim 30.

42. A method for the in vitro determination of ligands of a receptor no ligand of which is known or no ligand of which is known that can be used for specific affinity binding studies, comprising the use of a collection of claim 36.
